(19) United States
(12) Patent Application Publication
(10) Pub. No.: US 2009/0068670 A1
(43) Pub. Date: Mar. 12, 2009

(54) STRATIFIN DIRECTED DIAGNOSTICS FOR NEOPLASTIC DISEASE

(76) Inventors: Elias Georges, Laval (CA); Anne-Marie Bonneau, Laval (CA)

Correspondence Address: WILMERHALE/BOSTON, 60 STATE STREET, BOSTON, MA, 02109, US

(21) Appl. No.: 12/209,858

(22) Filed: Sep. 12, 2008

Related U.S. Application Data
(60) Provisional application No. 60/993,377, filed on Sep. 12, 2007.

(51) Int. Cl. C12Q 1/68; G01N 33/566

(52) U.S. Cl. 435/6; 435/7.1

(57) ABSTRACT

Disclosed are methods for diagnosing cancer in a test cell sample or fluid sample by detecting an increase in the level of expression of stratifin in the test cell sample or fluid sample as compared to the level of expression of stratifin in a control cell sample or fluid sample isolated from a normal subject.

This application claims the benefit of priority to U.S. Provisional Application No. 60/993,377, filed Sep. 12, 2007, the specification of which is incorporated by reference in its entirety.

### FIELD OF THE INVENTION

The present invention relates generally to the field of medicine. More specifically, the invention pertains to methods and devices for detecting the development of cancer in a subject.

### BACKGROUND OF THE INVENTION

Cancer is one of the deadliest illnesses in the United States. It accounts for nearly 600,000 deaths annually in the United States, and costs billions of dollars for those who suffer from the disease. This disease is in fact a diverse group of disorders, which can originate in almost any tissue of the body. In addition, cancers may be generated by multiple mechanisms including pathogenic infections, mutations, and environmental insults (see, e.g., Pratt et al. (2005) Hum Pathol. 36(8): 861-70). The variety of cancer types and mechanisms of tumorigenesis add to the difficulty associated with treating a tumor, increasing the risk posed by the cancer to the patient's life and wellbeing.

Cancers manifest abnormal growth and the ability to move from an original site of growth to other tissues in the body (hereinafter termed “metastasis”), unlike most non-cancerous cells. These clinical manifestations are therefore used to diagnose cancer because they are applicable to all cancers. Additionally, a cancer diagnosis is made based on identifying cancer cells by their gross pathology through histological and microscopic inspection of the cells. Although the gross pathology of the cells can provide accurate diagnoses of the cells, the techniques used for such analysis are hampered by the time necessary to process the tissues and the skill of the technician analyzing the samples. These methodologies can lead to unnecessary delay in treating a growing tumor, thereby increasing the likelihood that a benign tumor will acquire metastatic characteristics. It is thus necessary to accurately diagnose potentially cancerous growths as quickly as possible to avoid the development of a potentially life threatening illness.

One potential method of increasing the speed and accuracy of cancer diagnoses is the examination of genes as markers for neoplastic potential. Recent advances in molecular biology have identified genes involved in cell cycle control, apoptosis, and metabolic regulation (see, e.g., Isoldi et al. (2005) Mini Rev. Med. Chem. 5(7): 685-95). Mutations in many of these genes have also been shown to increase the likelihood that a normal cell will progress to a malignant state (see, e.g., Soejima et al. (2005) Biochem. Cell Biol. 83(4): 429-37). For example, mutations in p53, which is a well-known tumor suppressor gene, have been associated with aberrant cell growth leading to neoplastic potential (see Li et al. (2005) World J. Gastroenterol. 11(19): 2998-3001). Many mutations can affect the levels of expression of certain genes in the neoplastic cells as compared to normal cells.

There remains a need to identify an accurate and rapid means for diagnosing cancer in patients. Treatment efficacy would be improved by more efficient diagnoses of tissue samples. Furthermore, rapid diagnoses of cancerous tissues would allow clinicians to treat potential tumors prior to the metastasis of the cancer to other tissues of the body. Finally, a test that did not rely upon a particular technician's skill at identifying abnormal histological characteristics would improve the reliability of cancer diagnoses. There is, therefore, a need for new methods of diagnoses for cancer that are accurate, fast, and relatively easy to interpret.

### SUMMARY OF THE INVENTION

The subject matter disclosed herein is based, in part, upon the discovery that differential expression of stratifin at the protein and RNA levels occurs when a cell progresses to a neoplastic state. These expression patterns are therefore diagnostic for the presence of cancer in a cell sample. This discovery has been exploited to provide an invention that uses such patterns of expression to diagnose the presence of neoplastic cells in the cell sample.

In one aspect, a method of detecting a neoplasm is provided. The method comprises the step of obtaining a potentially neoplastic test cell sample and a non-neoplastic control cell sample. The method includes a step of detecting a level of expression of stratifin in the test cell sample, and detecting a level of expression of stratifin in the control cell sample. The method further includes a step of comparing the level of expression of stratifin in the test cell sample to the level of expression of stratifin in the control cell sample. The test cell sample is neoplastic if the level of expression of stratifin in the test cell sample is detectably greater than the level of expression of stratifin in the control cell sample.

In some embodiments, the method includes isolating cytoplasmic fractions from the test cell sample and the control cell sample, and then separately detecting the levels of expression of stratifin in the cytoplasmic fractions. In other embodiments, the method includes the level of expression of stratifin protein is detected by contacting the test cell sample and the control cell sample with a protein binding agent selected from the group consisting of antibody and stratifin binding portions thereof. In other embodiments, the method comprises detecting the level of protein binding agents bound to stratifin protein by detecting a detectable label such as immunofluorescent label, radiolabel, and chemiluminescent label.

In some embodiments, the protein-binding agent is immobilized on a solid support. In other embodiments, the level of expression of stratifin RNA is detected by contacting the test cell sample and the control cell sample with a nucleic acid binding agent selected from the group consisting of RNA, cDNA, cRNA, and RNA-DNA hybrids. In certain embodiments, the level of nucleic acid binding agent hybridized to stratifin RNA is detected by a detectable label such as an immunofluorescent label, a radiolabel, or a chemiluminescent label. In still other embodiments, the nucleic acid binding agent is immobilized on a solid support.

In some embodiments, the level of expression of stratifin in the test fluid sample is at least 1.5 times greater than the level of expression of stratifin in the control fluid sample. In other embodiments, the level of expression of stratifin in the test fluid sample is at least 2 times greater than the level of expression of stratifin in the control fluid sample. In still other embodiments, the level of expression of stratifin in the test fluid sample is at least 4 times greater than the level of expression of stratifin in the control fluid sample. In alternative embodiments, the level of expression of stratifin in the test fluid sample is at least 6 times greater than the level of expression of stratifin in the control fluid sample. In other embodiments, the level of expression of stratifin in the test fluid sample is at least 8 times greater than the level of expression of stratifin in the control fluid sample. In certain embodiments, the level of expression of stratifin in the test fluid sample is at least 10 times greater than the level of expression of stratifin in the control fluid sample. In some embodiments, the level of expression of stratifin in the test fluid sample is at least 20 times greater than the level of expression of stratifin in the control fluid sample.

In some embodiments, the test cell sample is obtained from a patient suffering from a metastasized lung neoplastic disease isolated from a tissue such as blood, bone marrow, spleen, lymph node, liver, thymus, kidney, brain, skin, gastrointestinal tract, eye, breast, and prostate. In other embodiments, the test cell sample is obtained from a patient suffering from non-small cell lung carcinoma.

In another aspect, the invention provides a kit for diagnosing or detecting neoplasia. The kit includes a first probe for the detection of stratifin.

In some embodiments, the probe for detecting stratifin is an anti-stratifin antibody or binding fragment thereof. In some embodiments, the probe detects stratifin present in the test cell sample if the patient is suffering from neoplastic disease. In some embodiments, the probe is immobilized on a solid support.

In still another aspect, a method of diagnosing cancer in a subject is provided. The method comprises the step of obtaining a test fluid sample (e.g., an ovarian fluid sample) and a control fluid sample from a non-neoplastic control sample (e.g., an ovarian fluid sample). The method includes a step of detecting a level of expression of stratifin in the test fluid sample, and detecting a level of expression of stratifin in the control fluid sample. The method further includes a step of comparing the level of expression of stratifin in the test fluid sample to the level of expression of stratifin in the control fluid sample. Cancer is diagnosed if the level of expression of stratifin in the test fluid sample is detectably greater than the level of expression of stratifin in the control fluid sample.

In some embodiments, the method includes detecting the level of expression of stratifin comprises isolating cellular cytoplasmic fractions from the test fluid sample and the control fluid sample, and separately detecting the level of expression of stratifin in the cellular cytoplasmic fractions. In other embodiments, the method includes the level of expression of stratifin protein is detected by contacting the test fluid sample and the control fluid sample with a protein binding agent selected from the group consisting of antibodies and binding fragments thereof. In other embodiments, the method includes the level of protein binding agents bound to stratifin protein is detected by a detectable label such as immunofluorescent label, radiolabel, and chemiluminescent label.

In still other embodiments, the level of expression of anti-stratifin antibody is detected in a test fluid sample and a control fluid sample. In certain embodiments, the level of expression of anti-stratifin antibody is detected in a serum sample isolated from a subject. In certain other embodiments, the level of expression of anti-stratifin antibody is detected using antibodies or fragments thereof. In particular embodiments, the antibodies or fragments thereof are operably linked to a detectable label such as an immunofluorescent label, radiolabel, and/or chemiluminescent label.

In some embodiments, the protein-binding agent is immobilized on a solid support. In other embodiments, the level of expression of stratifin RNA is detected by contacting the test fluid and the non-neoplastic ovarian control fluid with a nucleic acid binding agent such as RNA, cDNA, cRNA, and RNA-DNA hybrids. In certain embodiments, the level of nucleic acid binding agent hybridized to stratifin RNA is detected by a detectable label such as immunofluorescent label, radiolabel, and chemiluminescent label. In still other embodiments, the nucleic acid binding agent is immobilized on a solid support.

In some embodiments, the level of expression of stratifin in the test fluid sample is 1.5 times greater than the level of expression of stratifin in the control fluid sample. In other embodiments, the level of expression of stratifin in the test fluid sample is 2 times greater than the level of expression of stratifin in the control fluid sample. In still other embodiments, the level of expression of stratifin in the test fluid sample is 4 times greater than the level of expression of stratifin in the control fluid sample. In alternative embodiments, the level of expression of stratifin in the test fluid sample is 6 times greater than the level of expression of stratifin in the control fluid sample.

In other embodiments, the level of expression of stratifin in the test fluid sample is 8 times greater than the level of expression of stratifin in the control fluid sample. In certain embodiments, the level of expression of stratifin in the test fluid sample is 10 times greater than the level of expression of stratifin in the control fluid sample. In some embodiments, the level of expression of stratifin in the test fluid sample is at least 20 times greater than the level of expression of stratifin in the control fluid sample.

In some embodiments, the test fluid sample is from a patient suffering from a metastasized lung neoplastic disease isolated from a tissue such as blood, bone marrow, spleen, lymph node, liver, thymus, kidney, brain, skin, gastrointestinal tract, eye, breast, and prostate. In more embodiments, the test fluid sample is a patient suffering from non-small cell lung carcinoma. In still other embodiments, the test cell sample is a cell such as blood cells, bone marrow cells, spleen cells, lymph node cells, liver cells, thymus cells, kidney cells, brain cells, skin cells, gastrointestinal tract cells, eye cells, breast cells, prostate cells, uterine cells, and ovary cells.

In certain embodiments, the fluid sample is isolated from saliva, tears, urine, sweat, plasma, blood, or serum.

In another aspect, the invention provides a kit for diagnosing or detecting neoplasia. The kit includes a first probe for the detection of stratifin and a second probe for the detection of a neoplasia marker such as cytokeratin 7, cytokeratin 18, cytokeratin 19, and/or calveolin-1.

In some embodiments, the probe for detecting stratifin is an anti-stratifin antibody or stratifin-binding fragment thereof. In some embodiments, the probe detects stratifin present in the test fluid sample if the patient is suffering from ovarian neoplastic disease. In some embodiments, the probe is immobilized on a solid support. In some embodiments, the stratifin probe is a nucleic acid probe such as RNA, cDNA, cRNA, and RNA-DNA hybrids. In certain embodiments, the stratifin nucleic acid probe is complementary to at least a 20 nucleotide sequence of a nucleic acid sequence consisting of SEQ ID NO: 1.

In other embodiments, the probe binds to an anti-stratifin antibody. In particular embodiments, the probe is an anti-stratifin antibody or anti-stratifin antibody binding fragment thereof. The anti-stratifin antibody can be operably linked to a detectable label.

### BRIEF DESCRIPTION OF THE FIGURES

The foregoing and other objects of the present invention, the various features thereof, as well as the invention itself may be more fully understood from the following description, when read together with the accompanying drawings in which:

FIG. 1 is a graphic representation of nucleic acid microarray analyses of stratifin RNA expression in samples from ovarian cancer patients (OVT), breast cancer patients (BrT), and lung cancer patients (LT) as well as normal, tissue-matched subjects (OVN, BrN, and LN).

FIG. 2 is a tabular representation of screening results showing the ratio of stratifin RNA expression in samples obtained from patients suffering from non-small cell lung carcinoma (N=10) and lung samples from normal subjects (N=15), the ratios being generated by dividing the level of stratifin expression in the patients and normal subjects to the level of expression in a control, H23 tumor cell line.

FIG. 3 is a graphic representation of a scatter plot showing the ratios calculated in FIG. 2.

FIG. 4 is a tabular representation showing the diagnostic sensitivity and specificity of stratifin expression for non-small cell lung carcinoma.

FIG. 5 is a graphic representation of a ROC curve showing the diagnostic sensitivity and specificity of stratifin expression for non-small cell lung carcinoma.

FIG. 6 is a graphic representation of a scatter plot showing the ratio of expression of stratifin RNA in non-small cell lung carcinoma patients as compared to a normal subject reference pool, as well as the ratio of expression of stratifin RNA in normal subjects as compared to the normal RNA pool.

### DETAILED DESCRIPTION OF THE INVENTION

Patent and scientific literature referred to herein establishes knowledge that is available to those of skill in the art. The issued US patents, allowed applications, published foreign applications, and references, including GenBank database sequences, that are cited herein are hereby incorporated by reference to the same extent as if each was specifically and individually indicated to be incorporated by reference.

### 1.1. General

Methods and kits are disclosed for diagnosing, detecting, or screening a test sample, such as a fluid sample, for tumorigenic potential and neoplastic characteristics such as aberrant growth. In addition, the methods and compositions allow for the improved clinical management of tumors by providing a method that detects the expression level of a gene or genes identified as markers for cancer.

Typically, a gene will affect the phenotype of the cell through its expression at the protein level. Mutations in the coding sequence of the gene can alter its protein product in such a way that the protein does not perform its intended function appropriately. Some mutations, however, affect the levels of protein expressed in the cell without altering the functionality of the protein, itself. Such mutations directly affect the phenotype of a cell by changing the delicate balance of protein expression in a cell. Therefore, an alteration in a gene's overall activity can be measured by determining the level of expression of the protein product of the gene in a cell.

Accordingly, one aspect provides a method for diagnosing cancer in a cell. The method utilizes protein-targeting agents to identify proteins, such as stratifin, in a potentially cancerous cell sample or potentially cancerous serum or fluid sample. Increased levels of expression of particular protein markers in a cell or serum or fluid sample and a decreased expression level of other protein markers in a cell or serum or fluid sample indicate the presence of a neoplasm.

As used herein, “about” means a numeric value having a range of ±10% around the cited value. For example, a range of “about 1.5 times to about 2 times” includes the range “1.35 times to 2.2 times” as well as the range “1.65 times to 1.8 times,” and all ranges in between.

As used herein, the term “greater than” means more than, such as when the level of expression for a particular marker in test sample is detectably more than the level of expression for the same marker in a control sample. In these circumstances, expression analyses are qualitatively determined. The level of expression for a marker can also be determined quantitatively in test and control samples. In quantitative studies, the level of expression for a marker in a test sample is greater than the level of expression for the same marker in a control sample when the level of expression in the test sample is quantifiably determined to be at least about 10% more than the level of expression in the control sample.

As used herein, the term “protein-targeting agent” means a molecule capable of binding or interacting with a protein or a portion of a protein. Such binding or interactions can include ionic bonds, van der Waals interactions, London forces, covalent bonds, and hydrogen bonds. The target protein can be bound in a receptor-binding pocket, on its surface, or any other portion of the protein that is accessible to binding or interactions with a molecule. Protein-targeting agents include, but are not limited to, proteins, peptides, ligands, peptidomimetic compounds, inhibitors, organic molecules, aptamers, or combinations thereof.

As used herein, the term “inhibitor” means a compound that prevents a biomolecule, e.g., a protein, nucleic acid, or ribozyme, from completing or initiating a reaction. An inhibitor can inhibit a reaction by competitive, uncompetitive, or non-competitive means. Exemplary inhibitors include, but are not limited to, nucleic acids, proteins, small molecules, chemicals, peptides, peptidomimetic compounds, and analogs that mimic the binding site of an enzyme. In some embodiments, the inhibitor can be nucleic acid molecules including, but not limited to, siRNA that reduce the amount of functional protein in a cell.

As used herein, the term “tumorigenic potential” means ability to give rise to either benign or malignant tumors. Tumorigenic potential may occur through genetic mechanisms such as mutation or through infection with vectors such as viruses and bacteria.

The term “cancer” refers herein to a disease condition in which a tissue or cells exhibit aberrant, uncontrolled growth and/or lack of contact inhibition. A cancer can be a single cell or a tumor composed of hyperplastic cells. In addition, cancers can be malignant and metastatic, spreading from an original tumor site to other tissues in the body. In contrast, some cancers are localized to a single tissue of the body.

As used herein, a “cancer cell” is a cell that shows aberrant cell growth, such as increased, uncontrolled cell proliferation and/or lack of contact inhibition. A cancer cell can be a hyperplastic cell, a cell from a cell line that shows a lack of contact inhibition when grown in vitro, or a cancer cell that is capable of metastasis in vivo. In addition, cancer cells include cells isolated from a tumor or tumors. As used herein, a “tumor” is a collection of cells that exhibit the characteristics of cancer cells. Non-limiting examples of cancer cells include melanoma, ovarian cancer, ovarian cancer, renal cancer, osteosarcoma, lung cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, and thymoma. Cancer cells are also located in the blood at other sites, and include, but are not limited to, lymphoma cells, melanoma cells, sarcoma cells, leukemia cells, retinoblastoma cells, hepatoma cells, renal cancer cells, osteosarcoma cells, myeloma cells, glioma cells, mesothelioma cells, and carcinoma cells.

Cancer cells may also have the ability to metastasize to other tissues in the body. Metastasis is the process by which a cancer cell is no longer confined to the tumor mass, and enters the blood stream, where it is transported to a second site. Upon entering the other tissue, the cancer cell gives rise to a second situs for the disease and can take on different characteristics from the original tumor. Nevertheless, the new tumor retains characteristics from the tissue from which it derives, allowing for clinical identification of the type of cancer no matter where in the body a cancer cell or group of cells metastasizes. The process of metastasis has been studied extensively and is known in the art (see, e.g., Hendrix et al. (2000) Breast Cancer Res. 2(6): 417-22).

In certain embodiments of the invention, the cancer cell sample is obtained from a metastasized tumor or group of cells. The metastasized cells may be isolated from tissues including, but not limited to, blood, bone marrow, lymph node, liver, thymus, kidney, brain, skin, gastrointestinal tract, breast, and prostate.

The term “protein markers” as used herein means any protein, peptide, polypeptides, group of peptides, polypeptides or proteins expressed from a gene, whether chromosomal, extrachromosomal, endogenous, or exogenous, which may produce a cancerous or non-cancerous phenotype in the cell or the organism.

As used herein, “gene” means any deoxyribonucleic acid sequence capable of being translated into a protein or peptide sequence. The gene is a DNA sequence that may be transcribed into an mRNA and then translated into a peptide or protein sequence. Extrachromosomal sources of nucleic acid sequences can include double-strand DNA viral genomes, single-stranded DNA viral genomes, double-stranded RNA viral genomes, single-stranded RNA viral genomes, bacterial DNA, mitochondrial genomic DNA, cDNA or any other foreign source of nucleic acid that is capable of generating a gene product.

Protein markers can have any structure or conformation, and can be in any location within a cell, including on the cell surface. Protein markers can also be secreted from the cell into an extracellular matrix or directly into the blood or other biological fluid. Protein markers can be a single polypeptide chain or peptide fragments of a polypeptide. Moreover, they can also be combinations of nucleic acids and polypeptides as in the case of a ribosome. Protein markers can have any secondary structure combination, any tertiary structure, and come in quaternary structures as well.

One useful protein marker used to identify a neoplastic disease is stratifin protein. The primary and three-dimensional structure of stratifin is known in the art (see, e.g., Wang et al. (1998) Biochemistry 37:12727-12736). Examples of stratifin amino acid sequences include, but are not limited to, GenBank Accession Nos. NP_{—}006133, NP_{—}061224, CAB92118, CAM14836, AAH23552, AAH02995, AAH00995, and AAH00329.

As used herein, the term “test fluid sample” is a fluid that is obtained or isolated from a subject potentially suffering from a neoplastic disease. A fluid sample is isolated from urine, blood, lymph, pleural fluid, pus, marrow, cartilaginous fluid, saliva, seminal fluid, menstrual blood, and spinal fluid. Fluid samples can be isolated from tissues isolated from a subject. For instance, the tissues can be isolated from organs or tissues including, but not limited to, brain, kidney, blood, cartilage, lung, ovary, lymph nodes, salivary glands, breast, prostate, testes, uterus, skin, bone, and bone marrow. Fluid samples potentially include a neoplastic cell or group of cells. A test fluid sample can also be obtained from necrotic material isolated from a tumor or tumors. Such cell or group of cells may show aberrant cell growth, such as increased, uncontrolled cell proliferation and/or lack of contact inhibition. The test fluid sample can include, for example, a cancer cell that can be a hyperplastic cell, a cell from a cell line that shows a lack of contact inhibition when grown in vitro, or a cancer cell that is capable of metastasis in vivo.

As used herein, the term “test cell sample” refers to a cell, group of cells, or cells isolated from potentially cancerous tumor tissues. A test cell sample is one that potentially exhibits tumorigenic potential, metastatic potential, or aberrant growth in vivo or in vitro. A test cell sample can be isolated from tissues including, but not limited to, blood, bone marrow, spleen, lymph node, liver, thymus, kidney, brain, skin, gastrointestinal tract, eye, breast, and prostate.

As used herein, the term “non-neoplastic control cell sample” refers to a cell or group of cells that is exhibiting noncancerous normal characteristics for the particular cell type from which the cell or group of cells was isolated. A control cell sample does not exhibit tumorigenic potential, metastatic potential, or aberrant growth in vivo or in vitro. A control cell sample can be isolated from normal tissues in a subject that is not suffering from cancer. It may not be necessary to isolate a control cell sample each time a cell sample is tested for cancer as long as the nucleic acids isolated from the normal control cell sample allow for probing against the focused microarray during the testing procedure stratifin

In another aspect, the invention provides methods for diagnosing cancer in a test cell sample by detecting stratifin protein using a dipstick assay, Western blots, and Enzyme-Linked Immunosorbent Assays (“ELISA's”).

Cancer can also be detected using a protein microarray. The methods can be practiced using a microarray composed of capture probes affixed to a derivatized solid support such as, but not limited to, glass, nylon, metal alloy, or silicon. Non-limiting examples of derivatizing substances include aldehydes, gelatin-based substrates, epoxies, poly-lysine, amines and silanes. Techniques for applying these substances to solid surfaces are well known in the art. In useful embodiments, the solid support can be comprised of nylon.

As used herein, the term “capture probe” is intended to mean any agent capable of binding a gene product in a complex cell sample or fluid sample. Capture probes can be disposed on the derivatized solid support utilizing methods practiced by those of ordinary skill in the art through a process called “printing” (see, e.g., Schena et. al., (1995) Science, 270(5235): 467-470). The term “printing”, as used herein, refers to the placement of spots onto the solid support in such close proximity as to allow a maximum number of spots to be disposed onto a solid support. The printing process can be carried out by, e.g., a robotic printer. The VersArray CHIP Writer Prosystem (BioRad Laboratories) using Stealth Micro Spotting Pins (Telechem International, Inc, Sunnyvale, Calif.) is a non-limiting example of a chip-printing device that can be used to produce a focused microarray for this aspect. The capture probes may be antibodies, fragments thereof, or any other molecules capable of binding a protein (herein termed “protein capture probes”). These probes may be attached to a solid support at predetermined positions.

The level of expression of stratifin in the potentially cancerous test cell sample or potentially cancerous test fluid sample is compared to the level of expression of stratifin in a non-neoplastic control cell or control fluid sample of the same tissue type. If the expression of stratifin in the potentially cancerous cell or fluid sample is greater than the expression of stratifin in the non-neoplastic control cell or fluid sample, then cancer is indicated. In some embodiments, the test cell or fluid sample is tumorigenic if the level of expression of stratifin in the potentially cancerous cell or fluid sample is 1.5 times greater than the level of expression of stratifin in the non-neoplastic control cell or fluid sample. In some embodiments, the test cell or fluid sample is tumorigenic if the level of expression of stratifin in the potentially cancerous cell or fluid sample is at least 1.5 times greater than the level of expression of stratifin in the non-neoplastic control cell or fluid sample. The test cell or fluid sample may be tumorigenic if the level of expression of stratifin in the potentially cancerous cell or fluid sample is at least 2 times greater, at least 4 times greater, at least 6 times greater, between 8 and 12 times greater, at least 15 times greater, or at least 20 times greater than the level of expression of stratifin in the non-neoplastic control cell or non-neoplastic fluid sample.

In embodiments in which test tissue and cell samples are used, cell samples can be isolated from human tumor tissues using means that are known in the art (see, e.g., Vara et al. (2005) Biomaterials 26(18):3987-93; Iyer et al. (1998) J. Biol. Chem. 273(5):2692-7). For example, the cell sample can be isolated from the ovary of a human patient with ovarian cancer. Ovarian cancer cells can be obtained from other tissues as well, as in the case of metastatic ovarian cancer. Non-limiting sites of ovarian cancer-derived metastases can include, but are not limited to, ovarian, bone, blood, lung, skin, brain, adipose tissue, muscle, gastrointestinal tissues, hepatic tissues, and kidney. Alternatively, the cell test or control cell sample can be obtained from a cell line. Cell lines can be obtained commercially from various sources (e.g., American Type Culture Collections, Mannassas, Va.). Alternatively, cell lines can be produced using techniques well known in the art.

In addition, the cell sample can be a cell line. Cancer cell lines can be created by one with skill in the art and are also available from common sources, such as the ATCC cell biology collections (American Type Culture Collections, Mannassas, Va.).

In certain embodiments, cancer in tissues that are of mixed cellular populations such as a mixture of cancer cells and normal cells is detected. In such cases, cancer cells can represent as little as 40% of the tissue isolated for the present invention to determine that the cell sample is tumorigenic. For example, the cell sample can be composed of 50% cancer cells for the present invention to detect tumorigenic potential. Cell samples composed of greater than 50% tumorigenic cells can also be used in the present invention. It should be noted that cell samples can be isolated from tissues that are less than 40% tumorigenic cells as long as the cell sample contains a portion of cells that are at least 40% tumorigenic.

In some embodiments, levels of expression of housekeeping proteins are used to normalize the signal obtained between patients. As used herein, the term “housekeeping proteins” refers to any protein that has relatively stable or steady expression at the protein level during the life of a cell. Housekeeping proteins can be protein markers that show little difference in expression between cancer cells and normal cells in a particular tissue type. Examples of housekeeping proteins are well known in the art, and include, but are not limited to, isocitrate lyase, acyltransferase, creatine kinase, TATA-binding protein, hypoxanthine phosphoribosyl transferase 1, and guanine nucleotide binding protein, beta polypeptide 2-like 1 (see, e.g., Pandey et al. (2004) Bioinformatics 20(17): 2904-2910). In addition, the housekeeping proteins are used to identify the proper signal level by which to compare the cell sample signals between proteins from different or independent experiments.

Another aspect provides a method of diagnosing cancer in a fluid sample. In this method, expression of stratifin in the fluid sample is measured. Expression levels for stratifin can be determined using any techniques known in the art. Useful ways to determine such expression levels include, but not limited to, Western blot, protein microarrays, dipstick assays, and Enzyme-Linked Immunosorbent Assays (“ELISA”) (see, e.g., U.S. Pat. Nos. 6,955,896; 6,087,012; 3,791,932; 3,850,752; and 4,034,074). Such examples are not intended to limit the potential means for determining the expression of a protein marker in a cell sample. Expression levels of markers in or by potentially cancerous cell samples and normal control cell samples can be compared using standard statistical techniques known to those of skill in the art (see, e.g., Ma et al., (2002) Methods Mol. Biol. 196:139-45).

The fluid sample can be isolated from a human patient by a physician and tested for expression of stratifin using a dipstick or any other method that relies on a solid support, solid state binding, change in color, or electric current. In addition, the cancer cell sample can be isolated from an organism that develops a tumor or cancer cells including, but not limited to, mouse, rat, horse, pig, guinea pig, or chinchilla. Cell samples can be stored for extended periods prior to testing or tested immediately upon isolation of the cell sample from the subject. Cell samples can be isolated by non-limiting methods such as surgical excision, aspiration from soft tissues such as adipose tissue or lymphatic tissue, biopsy, or removed from the blood. These methods are known to those of skill in the art.

In certain embodiments, the level of expression of anti-stratifin antibodies in a fluid sample is detected. The level of expression of anti-stratifin antibodies in a cell sample is detected using ELISA, Western blot, or dot blot. The level of expression of anti-stratifin antibodies can be detected using antibodies or binding fragments thereof, which are specifically directed against anti-stratifin antibodies. Such useful antibody fragments include, but are not limited to, Fab, F(ab)₂, and Fv.

A normal or ovarian cancer cell sample can be isolated from a human patient by a physician and tested for expression of protein markers using a dipstick or any other method that relies on a solid support, solid state binding, change in color, or electric current. In addition, the cancer cell sample can be isolated from an organism that develops a tumor or cancer cells including, but not limited to mammals such as mouse, rat, horse, pig, guinea pig, or chinchilla. Cell samples can be isolated by non-limiting methods such as surgical excision, aspiration from soft tissues such as adipose tissue or lymphatic tissue, biopsy, or removed from the blood. These methods are known to those of skill in the art. Cell samples can be stored for extended periods prior to testing or tested immediately upon isolation of the cell sample from the subject.

### 1.2. Nucleic Acid Binding Agents

In another aspect, the method of detecting cancer includes detecting a level of expression of stratifin RNA in a test fluid sample (i.e., neoplastic test fluid sample) and comparing the level of expression of stratifin RNA detected in the test fluid sample to the level of expression of stratifin RNA detected in the non-neoplastic control fluid sample. If the level of expression of stratifin RNA is greater in the test fluid sample than in the non-neoplastic control fluid sample, then cancer is indicated.

In still another aspect, the method of detecting cancer includes detecting a level of expression of stratifin RNA in a test cell sample (i.e., neoplastic test fluid sample) and comparing the level of expression of stratifin RNA detected in the test cell sample to the level of expression of stratifin RNA detected in the non-neoplastic control cell sample. If the level of expression of stratifin RNA is greater in the test cell sample than in the non-neoplastic control cell sample, then cancer is indicated.

As used herein, “nucleic acid binding agent” means a nucleic acid capable of hybridizing with a particular target nucleic acid sequence. Nucleic acid binding agents include any structure that can hybridize with a target nucleic acid such as an mRNA. Nucleic acids can include, but are not limited to, DNA, RNA, RNA-DNA hybrids, siRNA, and aptamers. Moreover, any detectable labels can be used so long as the label does not affect the hybridizing of the nucleic acid with its targeting. Labels include, but are not limited to, fluorophores, chemical dyes, radiolabels, chemiluminescent compounds, colorimetric enzymatic reactions, chemiluminescent enzymatic reactions, magnetic compounds, and paramagnetic compounds.

Examples of stratifin nucleic acid sequences detected in the present invention include, but are not limited to, GenBank Accession Nos. NM_{—}006142, NM_{—}139323, NM_{—}018754, and NM_{—}006826.

In certain embodiments, a focused microarray can be used to detect the levels of expression of stratifin. The term “focused microarray” as used herein refers to a device that includes a solid support with capture probe(s) affixed to the surface of the solid support. In some embodiments, the focused microarray has nucleic acids attached to a solid support. The capture probes are directed to the diagnosis of a specific condition, e.g., chemotherapeutic drug resistance. Typically, the support consists of silicon, glass, nylon or metal alloy. Solid supports used for microarray production can be obtained commercially from, for example, Genetix Inc. (Boston, Mass.). Moreover, the support can be derivatized with a compound to improve nucleic acid association. Exemplary compounds that can be used to derivatize the support include aldehydes, poly-lysine, epoxy, silane containing compounds and amines. Derivatized slides can be obtained commercially from Telechem International (Sunnyvale, Calif.).

In the case of nucleic acid binding agents, nucleic acid sequences that are selected for detecting stratifin expression may correspond to regions of low homology between genes, thereby limiting cross-hybridization to other sequences. Typically, this means that the sequences show a base-to-base identity of less than or equal to 30% with other known sequences within the organism being studied. Sequence identity determinations can be performed using the BLAST research program located at the NIH website (www.ncbi.nlm.nih.gov/BLAST). Alternatively, the Needleman-Wunsch global alignment algorithm can be used to determine base homology between sequences (see Cheung et al., (2004) FEMS Immunol. Med. Micorbiol. 40(1):1-9). In addition, the Smith-Waterman local alignment can be used to determine a 30% or less homology between sequences (see Goddard et al., (2003) J. Vector Ecol. 28:184-9).

Expression levels for the stratifin can be determined using techniques known in the art, such as, but not limited to, immunoblotting, quantitative RT-PCR, microarrays, RNA blotting, and two-dimensional gel-electrophoresis (see, e.g., Rehman et al. (2004) Hum. Pathol. 35(11):1385-91; Yang et al. (2004) Mol. Biol. Rep. 31(4):241-8). Such examples are not intended to limit the potential means for determining the expression of a gene marker in a breast cancer fluid sample.

### 1.3. Protein-Targeting Agents

Protein marker expression is used to identify tumorigenic potential. Protein markers, such as stratifin, can be obtained by isolation from a cell sample, or a fluid sample, using any techniques available to one of ordinary skill in the art (see, e.g., Ausubel et. al., Current Protocols in Molecular Biology, Wiley and Sons, New York, N.Y., 1999). Isolation of protein markers, including stratifin, from the potentially tumorigenic cell sample, or from a fluid sample obtained from a patient potentially suffering or suffering from neoplastic disease, allows for the generation of target molecules, providing a means for determining the expression level of the protein markers in the potentially tumorigenic cell or fluid sample as described below. The protein markers, such as stratifin, can be isolated from a tissue or fluid sample isolated from a human subject. The stratifin and other protein markers can be isolated from a cytoplasmic fraction or a membrane fraction of the sample. Protein isolation techniques known in the art include, but are not limited to, column chromatography, spin column chromatography, and protein precipitation stratifin can be isolated using methods that are taught in, for example, Ausubel et al., Current Protocols in Molecular Biology, Vol. 1, John Wiley & Sons, Inc., (1993).

Protein-targeting agents are provided such as binding agents, e.g., antibodies or antigen binding fragments thereof. These embodiments are described in detail below. Other potential protein targeting agents include, but are not limited to, peptidomimetic compounds, peptides directed to the active sites of an enzyme, nucleic acids, nucleic acid aptamers.

Inhibitors can also be used as protein targeting agents to bind to protein markers. Useful inhibitors are compounds that bind to a target protein, and normally reduce the “effective activity” of the target protein in the cell or cell sample. Inhibitors include, but are not limited to, antibodies, antibody fragments such as “Fv,” “F(ab′)2,” “F(ab),” “Dab” and single chains representing the reactive portion of an antibody (“SC-Mab”), peptides, peptidomimetic compounds, and small molecules (see, e.g., Lopez-Alemany et al. (2003) Am. J. Hematol. 72(4): 234-42; Miles et al. (1991) Biochem. 30(6): 1682-91). Inhibitors can perform their functions through a variety of means including, but not limited to, non-competitive, uncompetitive, and competitive mechanisms. For instance, the triosephosphate isomerase 1 inhibitor N-hydroxy-4-phosphono-butanamide has been described previously (see, e.g., Verlinde et al (1989) Protein Sci. 1(12): 1578-84) and is useful.

Protein-targeting agents, including antibodies can also be conjugated to non-limiting materials such as magnetic compounds, paramagnetic compounds, proteins, nucleic acids, antibody fragments, or combinations thereof. Furthermore, antibodies can be disposed on an NPV membrane and placed into a dipstick. Antibodies can also be immobilized on a solid support at pre-determined positions such as in the case of a microarray. For instance, antibodies can be printed or cross-linked via their Fc regions to pre-derivatized surfaces of solid supports. In addition, antibodies can be cross-linked using bifunctional crosslinkers to a functionalized solid support. Such bifunctional crosslinking is well known in the art (see, e.g., U.S. Pat. Nos. 7,179,447; 7,183,373).

Crosslinking of proteins, such as antibodies, to a water-insoluble support matrix can be performed with bifunctional agents well known in the art including 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Bifunctional agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates can be employed for protein immobilization.

Protein-targeting agents can be detectably labeled. As used herein, “detectably labeled” means that a targeting agent is operably linked to a moiety that is detectable. By “operably linked” is meant that the moiety is attached to the protein-targeting agent by either a covalent or non-covalent (e.g., ionic) bond. Methods for creating covalent bonds are known (see, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y., 1999).

Accordingly, a “detectable label” is a moiety that can be sensed. Such labels can be, without limitation, fluorophores (e.g., fluorescein (FITC), phycoerythrin, rhodamine), chemical dyes, or compounds that are radioactive, chemiluminescent, magnetic, paramagnetic, promagnetic, or enzymes that yield a product that may be colored, chemiluminescent, or magnetic. The signal is detectable by any suitable means, including spectroscopic, photochemical, biochemical, immunochemical, electrical, optical or chemical means. In certain cases, the signal is detectable by two or more means. In certain embodiments, protein targeting agents include fluorescent dyes, radiolabels, and chemiluminescent labels, which are examples that are not intended to limit the scope of the invention (see, e.g., Gruber et al. (2000) Bioconjug. Chem. 11(5): 696-704).

For example, protein-targeting agents may be conjugated to Cy5/Cy3 fluorescent dyes. These dyes are frequently used in the art (see, e.g., Gruber et al. (2000) Bioconjug. Chem. 11(5): 696-704). The fluorescent labels can be selected from a variety of structural classes, including the non-limiting examples such as 1- and 2-aminonaphthalene, p,p′diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein).

### 1.4. Antibodies for Detection of Stratifin

Aspects utilize monoclonal and polyclonal antibodies as protein targeting agents directed specifically against stratifin protein. In certain embodiments, stratifin is used alone as a protein marker to diagnose cancer. Anti-stratifin protein antibodies, both monoclonal and polyclonal, for use in the invention are available from several commercial sources (e.g., Santa Cruz Biotechnology, Santa Cruz, Calif.; and Biogenesis, Inc., Kingston, N.H.). stratifin antibodies can be administered to a patient orally, subcutaneously, intramuscularly, intravenously, or interperitoneally for in vivo detection and/or imaging.

As used herein, the term “polyclonal antibodies” means a population of antibodies that can bind to multiple epitopes on an antigenic molecule. A polyclonal antibody is specific to a particular epitope on an antigen, while the entire pool of polyclonal antibodies can recognize different epitopes. In addition, polyclonal antibodies developed against the same antigen can recognize the same epitope on an antigen, but with varying degrees of specificity. Polyclonal antibodies can be isolated from multiple organisms including, but not limited to, rabbit, goat, horse, mouse, rat, and primates. Polyclonal antibodies can also be purified from crude serums using techniques known in the art (see, e.g., Ausubel, et al., Current Protocols in Molecular Biology, Vol. 1, pp. 4.2.1-4.2.9, John Wiley & Sons, Inc., 1996).

The term “monoclonal antibody”, as used herein, refers to an antibody obtained from a population of substantially homogenous antibodies, i.e., the individual antibodies comprising the population are identical except for possible naturally occurring mutations that may be present in minor amounts. Monoclonal antibodies are highly specific, being directed against a single antigenic site. By their nature, monoclonal antibody preparations are directed to a single specific determinant on the target. Novel monoclonal antibodies or fragments thereof mean in principle all immunoglobulin classes such as IgM, IgG, IgD, IgE, IgA, or their subclasses or mixtures thereof. Non-limiting examples of subclasses include the IgG subclasses IgG1, IgG2, IgG3, IgG2a, IgG2b, IgG3, or IgGM. The IgG subtypes IgG1/κ and IgG2b/κ are also included within the scope of the present invention. Antibodies can be obtained commercially from, e.g., BioMol International LP (Plymouth Meeting, Pa.), BD Biosciences Pharmingen (San Diego, Calif.), and Cell Sciences, Inc. (Canton, Mass.).

The monoclonal antibodies herein include hybrid and recombinant antibodies produced by splicing a variable (including hypervariable) domain of an anti-stratifin antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. (See, e.g., U.S. Pat. No. 4,816,567; Mage and Lamoyi, in Monoclonal Antibody Production Techniques and Applications, (Marcel Dekker, Inc., New York 1987, pp. 79-97). Thus, the modified “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method (see, e.g., Kohler and Milstein (1975) Nature 256:495) or can be made by recombinant DNA methods (U.S. Pat. No. 4,816,567). The monoclonal antibodies can also be isolated from phage libraries generated using the techniques described in the art (see, e.g., McCafferty et al. (1990) Nature 348:552-554).

Alternative methods for producing antibodies can be used to obtain high affinity antibodies. Antibodies can be obtained from human sources such as serum. Additionally, monoclonal antibodies can be obtained from mouse-human heteromyeloma cell lines by techniques known in the art (see, e.g., Kozbor (1984) J. Immunol. 133, 3001; Boerner et al., (1991) J. Immunol. 147:86-95). Methods for the generation of human monoclonal antibodies using phage display, transgenic mouse technologies, and in vitro display technologies are known in the art and have been described previously (see, e.g., Osbourn et al. (2003) Drug Discov. Today 8: 845-51; Maynard and Georgiou (2000) Ann. Rev. Biomed. ng. 2:339-76; U.S. Pat. Nos. 4,833,077; 5,811,524; 5,958,765; 6,413,771; and 6,537,809).

Aspects also utilize polyclonal antibodies for the detection of stratifin. They can be prepared by known methods or commercially obtained.

1.5. Detection of Stratifin from Biological Fluids

In another aspect, an assay is included for the detection of stratifin protein using a protein-targeting agent to bind to the stratifin protein. The stratifin protein typically is a peptide, polypeptide, protein, glycoprotein, or protiolipid. The protein-targeting agent can comprise antigens and antibodies thereto; haptens and antibodies thereto; and hormones, ligands, vitamins, metabolites and pharmacological agents, and their receptors and binding substances. The protein-targeting agent may be an immunologically-active polypeptide or protein or molecular weight between 1,000 Daltons and 10,000,000 Daltons, such as an antibody or antigenic polypeptide or protein, or a hapten of molecular weight between 100 Daltons and 1,500 Daltons. Protein-targeting agents can bind to stratifin protein that are obtained from biological fluids. As used herein, the term “biological fluids” means aqueous or semi-aqueous liquids isolated from an organism in which biological macromolecules may be identified or isolated. Biological fluids may be disposed internally as in the case of blood serum, bile, or cerebrospinal fluid. Biological fluids can be excreted as in the non-limiting cases of urine, saliva, sweat, tears, mucosal secretions, lacrimal secretions, seminal fluid, sperm, and sebaceous secretions.

For detection of markers in biological fluids, detection devices can be used that are in the form of a “dipstick.” Such devices are known in the art, and have been applied to detecting stratifin protein in serum and other biological fluids (see, e.g. U.S. Pat. No. 4,390,343). In some instances, a dipstick-type device can be comprised of analytical elements where protein-targeting agents, such as antibodies, inhibitors, organic molecules, peptidomimetic compounds, ligands, organic compounds, or combinations thereof, are incorporated into a gel. The gel can be comprised of non-limiting substances such as agarose, gelatin or PVP (see, e.g., U.S. Pat. No. 4,390,343). The gel can be contained within an analytical region for reaction with a protein marker.

The “dipstick” format (exemplified in U.S. Pat. Nos. 5,275,785, 5,504,013, 5,602,040, 5,622,871 and 5,656,503) typically consists of a strip of porous material having a biological fluid sample-receiving end, a reagent zone and a reaction zone. As used herein, the term “reagent zone” means the area within the dipstick in which the protein-targeting agent and the stratifin protein in the biological sample come into contact. By the term “reaction zone”, is meant the area within the dipstick in which an immobilized binding agent captures the protein-targeting agent/protein marker complex. As used herein, the term “binding agent” refers to any molecule or group of molecules that can bind, interact, or associate with a protein-targeting agent/protein marker complex.

In certain embodiments, the biological fluid sample is wicked along the assay device starting at the sample-receiving end and moving into the reagent zone. The protein marker(s) to be detected binds to a protein-targeting agent incorporated into the reagent zone, such as a labeled protein-targeting agent, to form a complex. For example, a labeled antibody can be the protein-targeting agent, which complexes specifically with the protein marker. In other examples, the protein-targeting agent can be a receptor that binds to a protein marker in a receptor:ligand complex. In yet other examples, an inhibitor is used to bind to a protein marker, thereby forming a complex with the protein marker targeted by the particular inhibitor. In some examples, peptidomimetic compounds are used to bind to stratifin protein to mimic the interaction of a protein marker with a normal peptide. In other examples, the protein-targeting agent can be an organic molecule capable of associating with the protein marker. In all cases, the protein-targeting agent has a label. The labeled protein-targeting agent-protein marker complex then migrates into the reaction zone, where the complex is captured by another specific binding partner firmly immobilized in the reaction zone. Retention of the labeled complex within the reaction zone thus results in a visible readout.

A number of different types of other useful assays that measure the presence of a protein market are well known in the art. One such assay is an immunoassay. Immunoassays may be homogeneous, i.e. performed in a single phase, or heterogeneous, where antigen or antibody is linked to an insoluble solid support upon which the assay is performed. Sandwich or competitive assays may be performed. The reaction steps may be performed simultaneously or sequentially. Threshold assays may be performed, where a predetermined amount of analyte is removed from the sample using a capture reagent before the assay is performed, and only analyte levels of above the specified concentration are detected. Assay formats include, but are not limited to, for example, assays performed in test tubes, wells or on immunochromatographic test strips, as well as dipstick, lateral flow or migratory format immunoassays.

A lateral flow test immunoassay device may be used in this aspect of the invention stratifin In such devices, a membrane system forms a single fluid flow pathway along the test strip. The membrane system includes components that act as a solid support for immunoreactions. For example, porous or bibulous or absorbent materials can be placed on a strip such that they partially overlap, or a single material can be used, in order to conduct liquid along the strip. The membrane materials can be supported on a backing, such as a plastic backing. The test strip includes a glass fiber pad, a nitrocellulose strip and an absorbent cellulose paper strip supported on a plastic backing.

Antibodies that specifically bind with the target protein marker are immobilized on the solid support. The antibodies can be bound to the test strip by adsorption, ionic binding, van der Waals adsorption, electrostatic binding, or by covalent binding, by using a coupling agent, such as glutaraldehyde. For example, the antibodies can be applied to the conjugate pad and nitrocellulose strip using standard dispensing methods, such as a syringe pump, airbrush, ceramic piston pump or drop-on-demand dispenser. A volumetric ceramic piston pump dispenser can be used to stripe antibodies that bind the analyte of interest, including a labeled antibody conjugate, onto a glass fiber conjugate pad and a nitrocellulose strip.

The test strip can be treated, for example, with sugar to facilitate mobility along the test strip or with water-soluble non-immune animal proteins, such as albumins, including bovine (BSA), other animal proteins, water-soluble polyamino acids, or casein to block non-specific binding sites.

### 1.6. Cancer Diagnosis and Prediction Analysis

Cancer diagnoses can be performed by comparing the levels of expression of stratifin in a potentially neoplastic cell sample to the levels of expression for a protein marker or a set of protein markers in a normal control cell sample of the same tissue type. Alternatively, the level of expression of stratifin in a potentially cancerous cell sample is compared to a reference pool of stratifin that represents the level of expression for stratifin in a normal control population (herein termed “training set”). The training set also includes the data for a population that has a known tumor or class of tumors. This data represents the average level of expression that has been determined for the neoplastic cells isolated from the tumor or class of tumors. It also has data related to the average level of expression for stratifin for normal cells of the same cell type within a population. In these embodiments, the algorithm compares newly generated expression data for stratifin from a cell sample isolated from a patient containing potentially neoplastic cells to the levels of expression for stratifin in the training set. The algorithm determines whether a cell sample is neoplastic or normal by aligning the level of expression for stratifin with the appropriate group in the training set. In certain embodiments, software for performing the statistical manipulations described herein can be provided on a computer connected by data link to a data generating device, such as a microarray reader.

Class prediction algorithms can be utilized to differentiate between the levels of expression of markers in a cell sample and the levels of expression of markers in a normal cell sample (Vapnik, The Nature of Statistical Learning Theory, Springer Publishing, 1995). Exemplary, non-limiting algorithms include, but are not limited to, compound covariate predictor, diagonal linear discriminant analysis, nearest neighbor predictor, nearest centroid predictor, and support vector machine predictor (Simon et al., Design and Analysis of DNA Microarray Investigations: An Artificial Intelligence Milestone., Springer Publishing, 2003). These statistical tests are well known in the art, and can be applied to ELISA or data generated using other protein expression determination techniques such as dot blotting, Western Blotting, and protein microarrays (see, e.g., U.S. Appln. No. 2005/0239079).

It should be recognized that statistical analysis of the levels of expression of protein markers in a cell sample to determine cancer state does not require a particular algorithm or set of particular algorithms. Any algorithm can be used in the present invention so long as it can discriminate between statistically significant and statistically insignificant differences in the levels of expression of protein markers in a cell sample as compared to the levels of expression of the same protein markers in a normal cell sample of the same tissue type.

In some embodiments, an increased level of expression of stratifin in the potentially cancerous cell sample, or fluid sample, indicates that cancer cells exist in the cell sample. The algorithm makes the class prediction based upon the overall levels of expression found in the cell sample as compared to the levels of expression in the training set.

The type of analysis detailed above compares the level of expression for stratifin in the cell sample to a training set containing reference pools of protein that are representative of a normal population and a neoplastic population. In certain embodiments, the training set can be obtained with kits that can be used to determine the level of expression of stratifin in a patient cell sample. Alternatively, an investigator can generate new training sets using protein expression reference pools that can be obtained from commercial sources such as Asterand, Inc. (Detroit, Mich.). Comparisons between the training sets and the cell samples are performed using standard statistical techniques that are well known in the art, and include, but are not limited to, the ArrayStat 1.0 program (Imaging Research, Inc., Brock University, St. Catherine's, Ontario, Calif.). Statistically significant increased levels of expression in the cell sample of stratifin indicate that the cell sample contains a cancer cell or cells with tumorigenic potential. Also, standard statistical techniques such as the Student T test are well known in the art, and can be used to determine statistically significant differences in the levels of expression for stratifin in a patient cell sample (see, e.g., Piedra et al. (1996) Ped. Infect. Dis. J. 15:1). In particular, the Student T test is used to identify statistically significant changes in expression using protein microarray analysis or ELISA analysis (see, e.g., Piedra et al. (1996) Ped. Infect. Dis. J. 15:1).

### 1.7. Protein Microarray

Protein microarrays can be prepared by methods disclosed in, e.g., U.S. Pat. Nos. 6,087,102, 6,139,831, and 6,087,103. In addition, protein-targeting agents conjugated to the surface of the protein microarray can be bound by detectably labeled protein markers isolated from a cell sample or a fluid sample. This method of detection can be termed “direct labeling” because the protein marker, which is the target, is labeled. In other embodiments, protein markers can be bound by protein-targeting agents, and then subsequently bound by a detectably labeled antibody specific for the protein marker. These methods are termed “indirect labeling” because the detectable label is associated with a secondary antibody or other protein-targeting agent. An overview of protein microarray technology in general can be found in Mitchell, Nature Biotech. (2002), 20:225-229, the contents of which are incorporated herein by reference.

### 1.8. Kits

Additionally, kits are provided for detecting neoplasms such as ovarian cancer in a cell or a fluid sample. The kits include targeting agents for the detection of stratifin. A patient that potentially has a tumor or the potential to develop a tumor (“in need thereof”) can be tested for the presence of a tumor or tumor potential by determining the level of expression of targeting agents in a cell or fluid sample derived from the patient.

The kit can include labeled binding agents capable of detecting stratifin in a biological sample, as well as means for determining the amount of stratifin in the sample, and means for comparing the amount of stratifin in the potentially cancerous sample with a standard (e.g., normal non-neoplastic control cells). The binding agents can be packaged in a suitable container. The kit can further comprise instructions for using the compounds or agents to detect stratifin. Such a kit can comprise, e.g., one or more antibodies, or fragments thereof as binding agents, that bind specifically to at least a portion of stratifin.

The kit can also include a probe for detection of housekeeping protein expression. These probes advantageously allow health care professionals to obtain an additional data point to determine whether a specific or general cancer treatment is working so stratifin levels can be used to monitor the success of cancer treatment. The probes can be any binding agents such as labeled antibodies, or fragments thereof, specific for the housekeeping proteins. Alternatively or additionally, the probes can be inhibitors, peptidomimetic compounds, peptides and/or small molecules.

Data related to the levels of expression of the selected protein markers in normal tissues and neoplasms can be supplied in a kit or individually in the form of a pamphlet, document, floppy disk, or computer CD. The data can represent patient pools developed for a particular population (e.g., Caucasian, Asian, etc.) and is tailored to a particular cancer type. Such data can be distributed to clinicians for testing patients for the presence of a neoplasm such as an ovarian cancer. A clinician obtains the levels of expression for a protein marker or set of protein markers in a particular patient. The clinician then compares the expression information obtained from the patient to the levels of expression for the same protein marker or set of protein markers that had been determined previously for both normal control and cancer patient pools. A finding that the level of expression for the protein marker or the set of protein markers is similar to the normal patient pool data indicates that the cell sample obtained from the patient is not neoplastic. A finding that the level of expression for the protein marker or the set of protein markers is similar to the cancer patient pool data indicates that the cell sample obtained from the patient is neoplastic.

### 1.9. Testing

The diagnostic methods according to the invention were tested for their ability to diagnose cancer in test cell samples isolated from human subjects suffering from ovarian cancer, lung cancer, prostate cancer, hepatic cancer, pancreatic cancer, breast cancer, leukemia, sarcoma, melanoma, renal cancer, colon cancer, and osteosarchma.

The expression levels of stratifin were analyzed for differential expression in ovarian samples by Western blotting and focused microarray. The testing and results are described in detail below in the Examples.

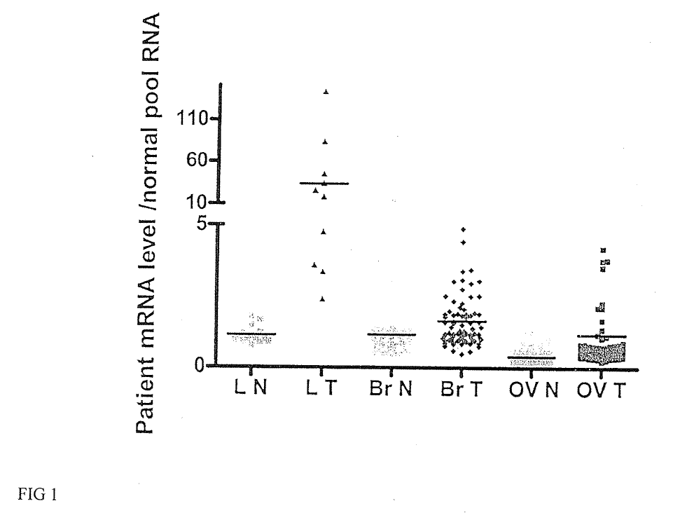

FIG. 1 shows the results of RNA expression experiments on normal samples isolated from normal subjects (OVN, BrN, and LN) and patient samples isolated from ovarian cancer patients (OVT), breast cancer patients (BrT), and lung cancer patients (LT). The cancer patients had increased levels of stratfin, as identified by anti-stratifin antibodies.

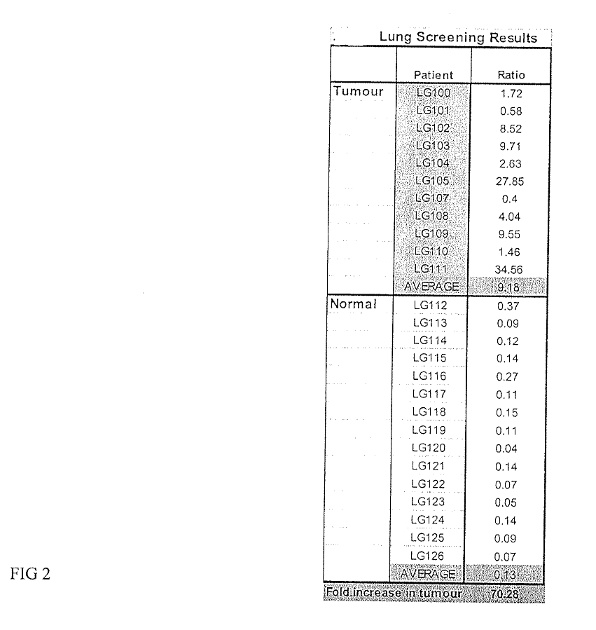

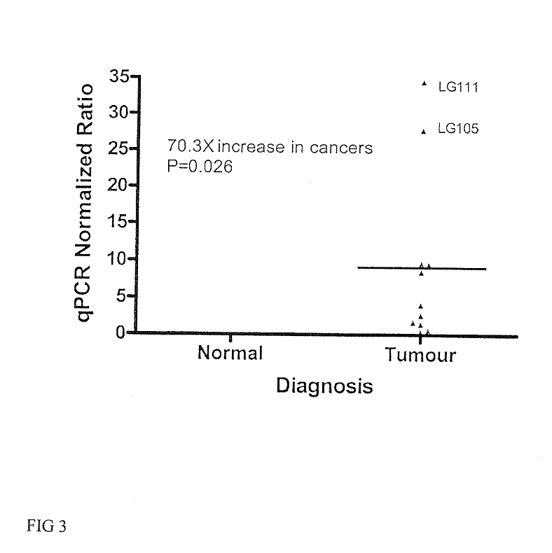

FIG. 2 shows the results of screening of non-small cell lung carcinoma in patients (designated LG100-LG111) and normal subjects (LG112-LG126). The data shows ratios generated by dividing stratifin RNA expression in patients and normal subjects by the level of expression identified in the tumor cell line H23 (FIG. 2). The ratios calculated in FIG. 1 are plotted on a scatter plot (FIG. 3).

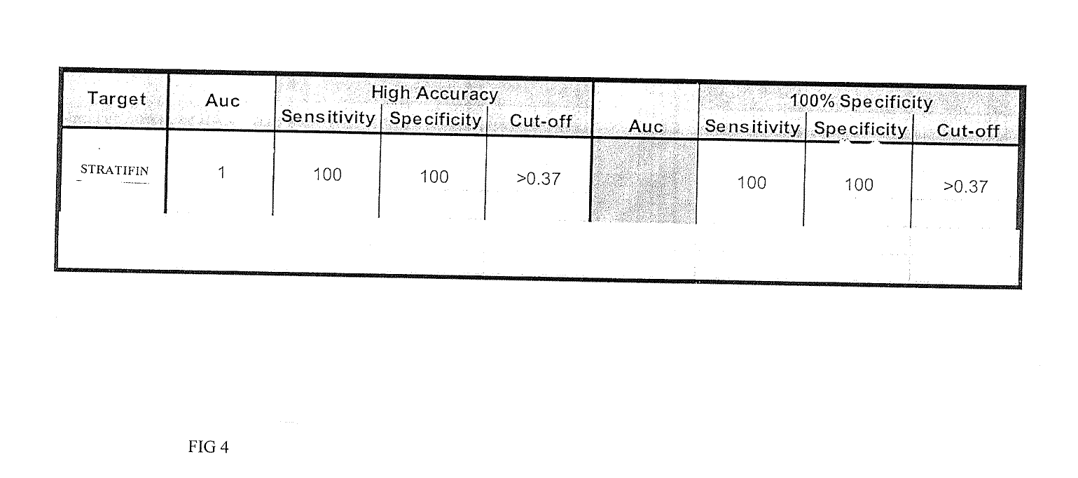

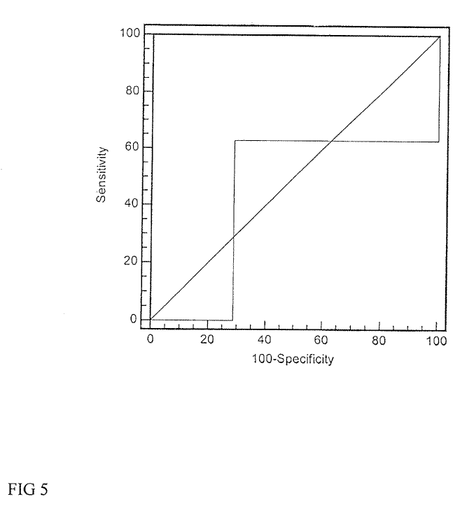

FIG. 4 shows that the sensitivity and specificity of stratifin as a diagnostic marker is 100%. This indicates that stratifin is a marker that is specific at identifying non-small cell lung carcinoma and sensitive at identifying the cancer. These results are shown in a ROC curve (FIG. 5).

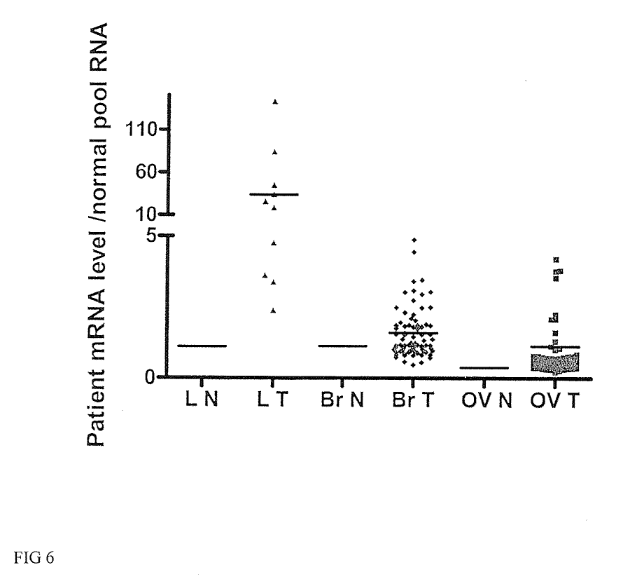

The level of stratifin RNA expression was also determined in patients suffering from non-small cell lung carcinoma, breast carcinoma, and ovarian carcinoma (FIG. 6). The level of stratifin expression was also determined in normal subjects. The levels of stratifin expression were divided by the level of stratifin RNA in a normal RNA pool. The ratios were then plotted (FIG. 6). As shown in FIG. 6, the levels of expression of stratifin in cancer patients was higher than the levels of expression in normal subjects. In particular, the non-small cell lung carcinoma patients were significant.

### EXAMPLES

Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Western Blot Analysis of Samples Isolated from Breast Cancer Patients and Normal Breast Subjects

### 1. Patient Samples and Normal Samples

Patient tissue samples are obtained from Asterand, Inc. (Detroit, Mich.), Clinomics Biosciences, Inc (Watervliet, N.Y.) and Biochain Institute, Inc. (Hayward, Calif.). The samples are isolated from normal breast and breast cancer samples, and are frozen into blocks of tissue. Protein cell extracts are then prepared from each block. Each patient included in the study is screened against the same normal total RNA pool in order to compare them together.

2. Western Blot Analysis of stratifin in Breast Cancer and Breast Normal Samples

For breast cell samples, human tissues are homogenized using a Polytron PT10-35 (Brinkmann, Mississauga, Canada) for 30 seconds at speed setting of 4 in the presence of 300 μl of 10 mM HEPES-Tris, pH 7.4, 150 mM NaCl, 1% Triton X-100, 1% sodium deoxycholic acid, 0.1% SDS, 1 mM EDTA and a cocktail of protease inhibitors from Roche Corp. (Laval, Qc, Canada). 40 μg of proteins from human breast cancer patients and normal breast subjects are used in SDS-PAGE gels. Samples are mixed with Laemmli buffer (250 mM Tris-HCl, pH 8.0, 25% (v/v) b-mercaptoethanol, 50% (v/v) glycerol, 10% (w/v) SDS, 0.005% (w/v) bromophenol blue), are heated for 5 mins. at 95° C. and are resolved in 12% SDS-polyacrylamide gels (SDS-PAGE). Proteins are then electro-transferred onto Hybond-ECL nitrocellulose membranes (Amersham Biosciences, Baie d'Urfé, Canada) for 90 mins. at 100 volts at RT (RT). Membranes are blocked for 1 hr. at RT in blocking solution (PBS containing 5% fat-free dry milk). Membranes are washed with PBS and incubated with the primary anti-stratifin polyclonal antibodies or monoclonal antibodies at the appropriate dilutions in blocking solution containing 0.02% sodium azide for 2 hrs. at RT. Antibodies are produced in house. PBS washing is performed, and the membranes are subsequently incubated for 1 hr. at RT with secondary anti-mouse, anti-rabbit or anti-goat antibodies labeled with horseradish peroxydase (Bio-Rad, Mississauga, Canada) diluted 1/3000 in PBS. Chemiluminescence detection is performed using the SuperSignal West Pico Chemiluminescent Substrate (Pierce, Rockford, Ill., USA) following the manufacturer's recommendations.

Stratifin expression is increased in tumor samples obtained from breast tumor patients as compared to normal samples isolated from normal subjects. Almost all normal subjects show nearly undetectable levels, or very low levels, of stratifin protein expression, while samples obtained from breast cancer patients show detectable levels, or increased levels, of stratifin. Stratifin protein expression is increased in tumor tissues as compared to normal tissues. The scatter plot shows that the majority of individual tumor samples have higher levels of stratifin expression as compared to the normal tissue samples.

### Example 2

### Preparation and Use of the Focused Microarray to Detect Stratifin in Samples Obtained from Normal Breast Subjects And Breast Cancer Patients

1. Total RNA Isolation and cDNA Labeling

Patient tissues samples were obtained from Asterand, Inc. (Detroit, Mich.), Clinomics Biosciences, Inc (Watervliet, N.Y.) and Biochain Institute, Inc. (Hayward, Calif.). stratifin Each patient included in the study was screened against the same normal total RNA pool in order to compare them together.

For breast cell samples, human tissues were homogenized using a Polytron PT10-35 (Brinkmann, Mississauga, Canada) for 30 seconds at speed setting of 4 in the presence of 300 μl of 10 mM HEPES-Tris, pH 7.4, 150 mM NaCl, 1% Triton X-100, 1% sodium deoxycholic acid, 0.1% SDS, 1 mM EDTA and a cocktail of protease inhibitors from Roche Corp. (Laval, Qc, Canada). Cell lysis, in the case of cell and tissue samples, and RNA extraction was done with the RNEasy kit, (#74104) (Qiagen, Inc., Valencia, Calif.) following the manufacturer's protocol. RNA was quantified by spectrophotometry using an Ultrospec 2000 spectrophotometer (Amersham-Biosciences, Corp., Piscataway, N.J.). RNA samples were dissolved in 10 mM Tris, pH 7.5 to determine the A_{260/280} ratios. Samples with ratios between 1.9 and 2.3 were kept for probe preparation, while samples with ratios lower than 1.9 were discarded. RNA samples were dissolved in 1 μl DEPC-H₂O for total nucleic acid quantification. Total RNA from control and treated samples was dried by speed vacuum using a Heto Vacuum centrifuge system (KNF Neuberger, Inc., Trenton, N.J.) at varying time intervals. The total RNA was resuspended in 10 μl of DEPC-H₂O and stored at −20° C. until the labeling reaction.

First strand cDNA labeling was accomplished using 1-15 μg total RNA (depending on the cell lines to be tested) for the resistant and the sensitive cell lines separately. Total RNA was incubated with 4 ng control positive Arabidopsis thaliana RNA, 3 μg of Oligo (dT)₁₂₋₁₈ primer (# Y01212) (Invitrogen, Corp., Carlsbad, Calif.), 1 μg PdN6 random primer (Amersham, #272166-01) for 10 min. at 65° C., and immediately put on ice for 1 min. The mixture was then diluted in 5× First strand buffer (250 mM Tris-HCl, pH 8.3; 375 mM KCl; 15 mM MgCl₂) containing 0.1 M DTT, 0.5 μM dNTPs mix (dTTP, dGTP, dATP) (Invitrogen, #10297-018), 0.05 μM dCTP (Invitrogen, #10297-018), 5 μM Cy3-dCTP (#NEL 576) (NEN Life Science/Perkin Elmer, Boston, Mass.), 2.5 μM Cy5-dCTP (#NEL 577) (NEN Life Science/Perkin Elmer, Boston, Mass.) and 400 units SuperScript III RNAse H-RT (Invitrogen, #18064-014). After incubating the reaction mixture for 5 min. at 25° C., the reaction mixture was incubated at 42° C. for 90 min. Finally, a total of 400 units of SuperScript II RNAse H⁻ RT (Invitrogen, #18064-014) were added and the reaction was incubated at 42° C. for another 90 min.

Digestion of the labeled cDNA with 5 units RNAse H (#M0297S) (NEB, Beverly, Mass.) and 40 units RNAse A (Amersham, #70194Y) was done at 37° C. for 30 min. The labeling probe was purified with the QIAquick PCR purification kit (Qiagen, Inc.) protocol with some modifications. Briefly, the reaction volume was completed to 50 μl with DEPC-H₂O and 2.7 μl of 12 M NaOAc pH 5.2 was added. The reaction was diluted with 200 μl PB buffer, put on the purification column, spun 15 sec. at 10 000 g, followed by 3 washes of 500 μl PE buffer (15 sec.; 10 000 g) and eluted 2 times in 50 μl DEPC-H₂O total (1 min.; 10 000 g). Frequency of incorporation and amount of cDNA labeled produced were evaluated for both labeled dCTPs by spectrophotometer (Ultrospec 2000, Pharmacia Biotech) at A_{260 nm}, A_{550 nm} and A_{650 nm}. The labeling material was dry by speed vacuum (Heto Vacuum centrifuge system, LaboPort) and resuspended in 3.75 μl H₂O total for both Cy5 (resistant cell line) and Cy3 reactions (sensitive cell line).

### 2. Capture Probe Preparation

Capture probes, approximately 68 nucleotides in length, corresponding to targets of interest were designed using sequences showing less identity base to base (<30%) with other coding sequences (cds) submitted to NCBI bank. The comparisons between sequences were done by BLAST research (www.ncbi.nlm.nih.gov/BLAST). For BioChip ver1.0 and ver2.0, a basic melting point temperature at a salt concentration of 50 mM Na⁺ (Tm) for each capture probe was calculated: the overall average was 76.97° C.+/−3.72° C. GC nucleotide content averaged 51.2%+/−9.4%. For the present invention, two negative controls (68 bp of the antisense cds of the BRCP and nucleophosmin targets) were synthesized.

The stratifin nucleic acid capture probe targeted ACRABP-II (gi#6382069) 481-548 bp of cds.

The capture probe was synthesized by the BRI Institute (Biotechnology Research Institute, Clear Water Bay, Kowloon, Hong Kong, China) with the Expedilite™ Synthesizer at a coupling efficiency of over 99.5% (Applied Biosystems, Foster City, Calif.). The oligonucleotides were verified by polyacrylamide gel electrophoresis. Oligonucleotide quantification was done by spectrophotometry at A_{260 nm}.

### 3. Printing of Capture Probes and Production of the Focused Microarray

Prior to printing of capture probes, different dilutions of Arabidopsis thaliana chlorophyll synthetase G4 DNA (undiluted solutions at 0.15 μg/μl and at 0.2 μg/μl; 1:2; 1:4; 1:8; 1:16) were printed on each grid as a positive control, and for normalization of results. Preparation of Arabidopsis thaliana control capture probes was performed as follows. Briefly, five micrograms of a Midi preparation using a HiSpeed™ Plasmid Midi kit (Qiagen, Inc.) of the Arabidopsis thaliana plasmid (gift of BRI) was digested with 40 units of Sac I enzyme (NEB) for 2 hr. at 37° C., purified with the QIAquick PCR purification kit (Qiagen) and verified by 1% agarose migration. In vitro transcription of 2 μg Sac I digestion was performed in 10× transcription buffer (400 mM Tris-HCl, pH 8.0; 60 mM MgCl₂; 100 mM DTT; 20 mM Spermidin) containing 2 μl of 10 mM NTP mix (Invitrogen), 20 units RNAse OUT (Invitrogen, #10777-019) and 50 units T7 RNA polymerase (NEB) for approximately 2 hr. to 30 hr. at 37° C. The reaction was then treated with 2 units DNAse I (Invitrogen) in 10×DNAse buffer (200 mM Tris-HCl pH 8.4; 20 mM MgCl₂; 500 mM KCl) for 15 min. at 37° C. The RNA was cleaned with the RNEasy kit (Qiagen) and quantified by spectrophotometry using an Ultrospec 2000 (Amersham Biosciences, Corp. Piscataway, N.J.).

After the control capture probes were generated and printed, the capture probes complementary to marker genes from the cancer cell samples were printed at concentrations of 25 μM in 50% DMSO on CMT-GAPS II Slides (#40003) (Corning, 45 Nagog Park, Acton, Mass.) by the VersArray CHIP Writer Prosystems (BioRad Laboratories) with the Stealth Micro Spotting Pins (#SMP3) (Telechem International, Inc., Sunnyvale, Calif.). Each capture probe was printed in triplicate on duplicate grids. Buffer and Salmon Testis DNA (Sigma D-7656) were also printed for the BioChip analysis step. After printing was completed, the slides were dried overnight by incubation in the CHIP Writer chamber. Chips were then treated by UV (Stratagene, UV Stratalinker) at 600 mJoules and baked in an oven for 6-8 hr.

### 4. Quality Control of Focused Microarray

Prior to testing the invention on cancer cell samples, the focused microarray was tested at the BRI Institute (Kowloon Bay, Hong Kong). One slide for each printed batch was quality control tested using a terminal deoxynucleotidyl transferase (Tdt)-mediated nick end labeling assay protocol (see, e.g., Yeo et. al., (2004) Clin. Cancer Res. 10(24): 8687-96). Additionally, controls were performed to verify the specificity of the hybridization using three independent grids on the same focused microarray.

As a first quality control, a test was done by the BRI Institute on one slide for each batch printed with the following Tdt transferase protocol. Briefly, the slide was prehybridized in a Hybridization Chamber (#2551) (Corning, Inc., Life Sciences, 45 Nagog Park, Acton, Mass.) with 80 μl of preheated prehybridization buffer (5×SSC (750 mM NaCl; 75 mM sodium citrate); 0.1% SDS; 1% BSA (Sigma, #A-7888) at 37° C. for 30 min. Slides were washed in 0.1×SSC (15 mM NaCl; 1.5 mM sodium citrate) and air-dried. 50 μl of TdT reaction mixture [5×TdT buffer (125 mM Tris-HCl, pH 6.6, 1 M sodium cacodylate, 1.25 mg/ml BSA); 5 mM CoCl₂; 1 mM Cy3-dCTP (NEN Life Science, NEL 576); 50 units TdT enzyme (#27-0730-01) (Amersham BioSciences)], was added to the entire area of the BioChip. The slide was incubated in the Hybridization Chamber for 60 min. at 37° C. following by a first wash in 1×SSC (150 mM NaCl; 15 mM sodium citrate)/0.2% SDS (preheated at 37° C.) for 10 min., a second wash of 5 min. in 0.1×SCC (15 mM NaCl; 1.5 mM sodium citrate)/0.2% SDS at RT and finally a last wash of 5 min. at RT in 0.1×SSC (15 mM NaCl; 1.5 mM sodium citrate). The slide was scanned with the ScanArray™ Lite MicroArray Scanner (Packard BioSciences, Perkin Elmer, San Jose, Calif.).

As a second quality control step, the PARAGON™ DNA Microarray Quality Control Stain kit (Molecular Probes) was incubated with the microarray according to the manufacturer's recommendations.

5. Focused Microarray Hybridization with Labeled cDNA Probes

Focused microarray slides were pre-washed before the prehybridization step as follows. First, slides were washed for 20 min. at 42° C. in 2×SSC (300 mM NaCl; 30 mM sodium citrate)/0.2% SDS under agitation. The second wash was for 5 min. at RT in 0.2×SSC (30 mM NaCl, 3 mM Sodium citrate) under agitation, and then followed by a wash for 5 min. at RT in DEPC-H₂O with agitation. The slides were spin dried at 1000 g for 5 min. and prehybridized in Dig Easy Hyb Buffer (#1,603,558) (Roche Diagnostics Corporation, Indianapolis, Ind.) containing 400 μg Bovine Serum Albumin (Roche, #711,454) at 42° C. in humid chamber for 3 hr. then washed 2 times in DEPC-H₂O, and once in Isopropanol (Sigma, 1-9516) and spun dry at 1000 g for 5 min.

To the mixed Cy5/Cy3 probe, 15 μg Baker tRNA (#109,495) (Roche Diagnostics Corp., Indianapolis, Ind.) and 1 μg Cot-1DNA (Roche, #1,581,074) were added and the probe was incubated 5 min. at 95° C., put on ice for 1 min., and diluted with 14 μl Dig Easy Hyb buffer (Roche, #1,603,558). After a 2 min. spin at 100 g, the probe was incubated at 42° C. for at least 5 min.

The three supergrids on the slide were separated by a Jet-Set Quick Dry TOP Coat 101 line (#FX268) (L'Oreal, Paris, FR). Each probe was added to its respective supergrid and covered by a preheated (42° C.) coverslip (Mandel, #S-104 84906). The slide was incubated at 42° C. in humid chamber for at least 15 hr.

The coverslips were removed by dipping in 1×SSC (150 mM NaCl; 15 mM sodium citrate)/0.2% SDS solution preheated at 50° C.). The slide was washed three times for 5 min. with agitation in 1×SSC (150 mM NaCl; 15 mM sodium citrate)/0.2% SDS solution preheated at 50° C.), and then washed three times with agitation in 0.1×SSC (15 mM NaCl; 1.5 mM sodium citrate)/0.2% SDS solution preheated at 37° C.). Finally, the slide was washed once in 0.1×SSC (15 mM NaCl; 1.5 mM sodium citrate) with agitation for 5 min. The slide was dipped several times in DEPC-H₂O and spun dry at 1000 g for 5 min.

### 6. Scanning and Statistical Analysis

The slides were scanned with a ScanArray™ Lite MicroArray Scanner (Packard BioSciences, Perkin Elmer, San Jose, Calif.) and the analysis was performed with a QuantArrayR Microarray Analysis software version 3.0 (Packard BioSciences, Perkin Elmer, San Jose, Calif.).

The QuantArray® data results were analyzed according to the following procedures. All analysis of the results was performed with the spot background subtracted values for Cy5 and Cy3. Spots with lower signal ratio to noise lower than 1.5 were discarded. Normalization of the ratios with the spike positive control (Arabidopsis thaliana) was done to have a ratio equal to one for that control on each slide. Slides were discarded on which the negative and/or positive controls did not work. Also, slides were discarded with high background and with different mean no offset correction (ArrayStat software). Mean for each target was calculated with at least six different experiments (including two reciprocal labeling reactions), each experiment using different total RNA preparations. Statistical analysis was accomplished with the ArrayStat 1.0 (Imaging Research Inc., Brock University, St. Catherine's, Ontario, Calif.). A log transformation of the ratio data is followed by a Student T test for two independent conditions using a proportional model without offsets at a p<0.05 threshold. Significant increases (ratio Cy5/Cy3 higher than 1.5) or decreases (ratio Cy5/Cy3 lower than 0.5) were considered to be significant if the p value was lower than 0.05.

### 7. Results.

mRNA expression correlated with stratifin protein expression. Increased levels of stratifin mRNA were detected in tumor samples (BrT) obtained from patients suffering from breast cancer as compared to samples from normal subjects (BrN) (FIG. 1). Tumor samples from patients suffering from breast cancer had higher levels of stratifin RNA expression than did samples from normal subjects (FIG. 1).

### Example 3

### Western Blot Analysis of Samples Isolated from Lung Cancer Patients and Normal Lung Subjects

### 1. Patient Samples and Normal Samples

Patient lung tissues and pleural fluid samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA pool in order to compare them together.

2. Western Blot Analysis of stratifin in Lung Cancer and Lung Normal Samples

Fluid samples are prepared as described in Example 1. Lung tissue samples are prepared as described in Example 1.

### 3. Results.

Stratifin expression is significantly increased in cell and fluid samples obtained from lung cancer patients as compared to cell and fluid samples isolated from normal subjects. All normal subjects show undetectable, or nearly undetectable, levels of stratifin protein expression, while samples obtained from lung cancer patients show detectable levels of stratifin.

### Example 4

### Preparation and Use of Focused Microarray to Detect Stratifin in Samples Obtained From Normal Lung Subjects and Lung Cancer Patients

1. Total RNA Isolation and cDNA Labeling

Patient lung tissue samples and pleural fluid samples were obtained from Asterand, Inc. (Detroit, Mich.), Clinomics Biosciences, Inc (Watervliet, N.Y.) and Biochain Institute, Inc. (Hayward, Calif.). Each patient included in the study was screened against the same normal total RNA pool in order to compare them together.

Fluid samples were prepared as described in Example 2. Lung tissue samples were homogenized as described in Example 2.

### 3. Results.

mRNA expression correlates with stratifin protein expression. Increased levels of stratifin mRNA were detected in samples obtained patients suffering from lung cancer (LT) as compared to samples from normal subjects (LN) (FIG. 1). Samples from patients suffering from lung cancer showed up to six times higher levels of stratifin RNA expression than did samples from normal subjects (FIG. 1).

### Example 5

### Classification of Cell Samples Isolated from Lung Normal Subjects and Non-Small Cell Lung Carcinoma Patients Using Quantitative RT-PCR

### 1. Patient Samples and Normal Samples

Total RNA was extracted from snap frozen patient tissue samples of Asterand, Inc., Clinomics Biosciences, Inc. or Biochain Institute, Inc. with the Trizol Reagent kit (Gibco-BRL, Carlsbad, Calif.) using the recommended extraction procedures. Eleven patients were tested in Q-PCR and fifteen normal subjects. Standard clinical and pathological reports were available for each cancer patient included in this study. Total RNA was treated with RNA-free DNAse I (New England BioLabs, Beverly, Mass.), and was purified with the RNEasy kit (Qiagen, Hilden, Germany). RNA samples were visualized on an Agilent 2100 BioAnalyzer (Agilent Technologies, Foster City, Calif.) for purity and integrity.

### 2. RNA Primer Design and Testing

The design of the primers used during for Quantitative RT-PCR required analysis to ensure proper annealing would occur so as to provide proper products. Briefly, several potential primer pairs were analyzed to determine potential hairpin formation and formation of dimer pairs using PrimerQuest® software according to manufacturer's instructions (Integrated DNA Technologies, Coralville, Iowa).

Upon determination of the appropriate primer pairs to use for the Quantitative RT-PCR analysis, the optimal concentration for each primer pair was determined. Briefly, RT-PCR was performed as described below using all primer pairs at varying concentrations between 100 nM and 300 nM. Ct values were determined for each concentration as described below.

### 3. Total RNA Reaction Mixtures Utilized in the Present Study

One hundred ng of total RNA, as measured by the NanoDrop® ND-1000 spectrophotometer (NanoDrop Technologies, Wilmington, Del.), was used for reverse transcription into cDNA. Briefly, tRNA was mixed with 500 ng oligodT₁₈, 250 ng pdN₆ random primers, and 10 pg Arabidopsis RNA (internal control) to a final volume of 10 μl. The mixture was heated 10 min. at 65° C. and quickly transferred to ice for 2 min. The cDNA synthesis solution (50 mM Tris-HCl, pH 8.3; 75 mM KCL; 3 mM MgCl₂; 10 mM DTT; 1 mM dNTP; 200 unit of SSIII enzyme) was added and incubated for 5 min. at 25° C., followed by 1.5 hr. at 50° C. The mixture was then centrifuged, heated for 5 min. at 95° C., and centrifuged a second time. The samples were placed on ice until further use.

4. First Strand cDNA Reaction

Quantitative RT-PCR experiments were done on a Stratagene Mx3005 QPCR system (Straragene, La Jolla, Calif.) using SYBRGREEN (Stratagene, La Jolla, Calif.) binding dye. Specific primer sets were designed using the Primer Quest software (Integrated DNA Technologies, Coralville, Iowa) (see Table 7 for primers). For each transcript set, the optimum concentration was determined and standard curves were generated using six dilutions of a cDNA sample from a normal pool. To confirm that the amplification occurred on the targeted sequences, the dissociation curves were examined for the presence of a single sharp peak at the melting temperature of the amplicon. Typical standard curve included 0, 1/5, 1/10, 1/25, 1/50, 1/250, and 1/500 dilutions of cDNA (Lung normal pool).

The cDNA(1/25) was mixed with Brillant Quantitative RT-PCR MasterMix solution (Stratagene, La Jolla, Calif.), which included 100 nM of each primer (forward and reverse). The mixture was placed in 96-wells plate. PCR reactions consisted of 40 cycles of denaturation for 30 seconds at 95° C., 40 cycles of annealing for 1 min. at 60° C., and 40 cycles of extension for 30 seconds at 72° C., after an initial denaturation step at 95° C. for 10 min. Experiment were performed in duplicate for each data point and repeated twice. The Quantitative RT-PCR experiments were performed with the arabidopsis internal control to determine the efficiency of the PCR reaction. A standard curve of normal lung pool was also compared to the lung patient samples.

### 5. Standard Curves

The dilution sets of Normal pool of RNA used for the standard curve were based on preliminary results were serial dilutions of cDNA of the BRpool were used with all the ABp targets in a Real-time PCR reaction. The dilution sets are <1/5> or <1/10>, <1/50>, <1/250> and <1/500>. The dilutions sets that, all together, were giving efficiency between 90% and 110% were chosen. Eighteen PCR runs (19 for Arabidopsis) were performed to screen all the patients twice. On certain occasions, dilution sets or replicates had to be removed to obtain an acceptable curve. PCR efficiency for each primer pair was determined.

A primer pair was considered “perfect” if the efficiency is between 90% and 110%. The results indicate that the primers have efficiencies that are within the acceptable ranges.

### 6. Statistical Analysis

At the end of each run, the computer provided all PCR amplification curves. The Ct value was used for the analysis of the results. This value corresponded to the cycle number, in the linear phase of the amplification, where the fluorescence reaches a given threshold. The amount of target at this cycle was given by Equation 1:

Xₙ=═X₀×2ⁿ n=number of cycles

The comparative evaluation, based on the cycle threshold (Ct) of stratifin normalize to the arabidopsis control, was used to evaluate the relative gene expression.

The ratio (sample/Normal pool) was determined following the equation of Pfaffl that takes into consideration the efficiency of the PCR reaction (Paffl 2001):

$$Ratio = \frac{{( 1 + {E}_{target} )}^{dCttarget ( Control - Sample )}}{{( 1 + {E}_{ref} )}^{dCtref ( Control - Sample )}}$$

Ratio: sample/Normal pool
E: efficiency of the PCR reaction for the given primer set (based on the slope of the standard curve)
Target: ABp target
Control: Normal pool
Ref: Spiked Arabidopsis
dCt: Ct of the control (Normal pool)−Ct of the target

### 5. Results

The results of the validation of focused microarray data are described in FIG. 6. An increased level of expression of stratifin is a marker of non-small cell lung carcinoma (FIG. 6). The increased level of expression of stratifin RNA in non-small cell lung carcinoma as compared to normal samples is predictive of cancer. All patients have an increased level of expression of stratifin as compared to normal subjects (FIG. 6).

### Example 6

### Classification of Cell Samples Isolated from Lung Normal Subjects and Non-Small Cell Lung Carcinoma Patients Using Quantitative RT-PCR

### 1. Patient Samples and Normal Samples

The level of stratifin expression was identified in non-small cell lung carcinoma patients and normal subjects utilizing the same techniques as in Example 5. Patient material was purchased from Asterand, Inc. (Detroit, Mich.), Clinomics Biosciences, Inc (Watervliet, N.Y.) and Biochain Institute, Inc. (Hayward, Calif.). Eleven patients and fifteen normal subjects were tested in Q-PCR. Ratios were generated by determining the level of expression of stratifin in the lung tumor cell line H23. The ratios were generated using the calculations disclosed in Example 5.

### 2. Results

As shown in FIGS. 2-5, increased stratifin expression is diagnostic of non-small cell lung carcinoma. Increased levels of stratifin mRNA were detected at 70 times greater levels in non-small cell lung carcinoma patients as compared to normal subjects (FIGS. 2-3). The increased expression of stratifin was 100% specific and sensitive for non-small cell lung carcinoma (FIGS. 4-5). These data establish that increased stratifin expression is diagnostic of non-small cell lung carcinoma.

### Example 7

### Western Blot Analysis of Samples Isolated from Ovarian Cancer Patients and Normal Ovarian Subjects

### 1. Patient Samples and Normal Samples

Patient ovarian tissues and pleural fluid samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA pool in order to compare them together.

2. Western Blot Analysis of stratifin in Ovarian Cancer and Ovarian Normal Samples

Fluid samples are prepared as described in Example 1. Ovarian tissue samples are prepared as described in Example 1.

### 3. Results.

Stratifin expression is significantly increased in cell and fluid samples obtained from ovarian cancer patients as compared to cell and fluid samples isolated from normal subjects. All normal subjects show undetectable, or nearly undetectable, levels of stratifin protein expression, while samples obtained from ovarian cancer patients show detectable levels of stratifin.

### Example 8

Preparation and Use of Focused Microarray to Detect Stratifin in Samples Obtained From Normal Ovarian Subjects and Ovarian Cancer Patients

1. Total RNA Isolation and cDNA Labeling

Patient ovarian tissue samples and pleural fluid samples were obtained from Asterand, Inc. (Detroit, Mich.), Clinomics Biosciences, Inc (Watervliet, N.Y.) and Biochain Institute, Inc. (Hayward, Calif.). Each patient included in the study was screened against the same normal total RNA pool in order to compare them together.

Fluid samples were prepared as described in Example 2. Ovarian tissue samples were homogenized as described in Example 2.

### 2. Results.

mRNA expression correlates with stratifin protein expression. Increased levels of stratifin mRNA were detected in samples obtained patients suffering from ovarian cancer (OVT) as compared to samples from normal subjects (OVN) (FIG. 1). Samples from patients suffering from ovarian cancer showed about two times higher levels of stratifin RNA expression than did samples from normal subjects (FIG. 1).

### Example 9

### Classification of Cell Samples Isolated from Ovarian Normal Subjects and Ovarian Adenocarcinoma Patients Using Quantitative RT-PCR

### 1. Patient Samples and Normal Samples

Patient material was purchased from Asterand, Inc. (Detroit, Mich.), Clinomics Biosciences, Inc (Watervliet, N.Y.) and Biochain Institute, Inc. (Hayward, Calif.). Sixty-two patients and seventy-seven were tested in Q-PCR.

The level of stratifin expression was identified in ovarian adenocarcinoma patients and normal subjects utilizing the same techniques as in Example 5. The ratios were generated using the calculations disclosed in Example 5.

### 2. Results

As shown in FIG. 6, increased stratifin expression is identified in certain ovarian adenocarcinoma patients as compared to normal subjects.

### Example 10

### Western Blot Analysis of Samples Isolated from Leukemia Patients and Normal Subjects

### 1. Patient Samples and Normal Samples

Patient marrow tissues and blood are obtained from Asterand, Inc. (Detroit, Mich.), Clinomics Biosciences, Inc (Watervliet, N.Y.) and Biochain Institute, Inc. (Hayward, Calif.). stratifin Each patient included in the study is screened against the same normal total RNA pool in order to compare them together.

### 2. Western Blot Analysis of Stratifin in Leukemia and Normal Samples

Blood samples are prepared by isolating blood from leukemia patients. The blood samples are fractioned initially to remove red-blood cells. The remaining samples containing all white blood cell are further fractionated by FACS sorting based on size defractions and/or using surface specific monoclonal antibodies. Purified cells are then lysed in lysis buffer as in the above examples. Quantified cell lysates from leukemia samples and normal blood cells are then resolved on SDS-PAGE and prepared for Western blotting to probe for stratifin.

### 3. Results.

The results of expression analyses for the protein markers is that stratifin expression is significantly increased in cell and fluid samples obtained from tumor patients as compared to cell and fluid samples isolated from normal subjects. All normal subjects show undetectable, or nearly undetectable, levels of stratifin protein expression, while samples obtained from leukemia patients show detectable levels of stratifin.

### Example 111

### Preparation and Use of Focused Microarray to Detect Stratifin in Samples Obtained From Normal Subjects and Leukemia Patients

1. Total RNA Isolation and cDNA Labeling

Patient marrow tissues and blood are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA pool in order to compare them together.

Blood samples are prepared as described in Example 7. For leukemia tissue samples, human marrow tissues are homogenized and prepared for analysis following procedures described in Example 1.

First strand cDNA labeling, cDNA digestion, capture probe preparation and focused microarray preparation are accomplished using procedures described in Example 2. In addition, quality control and focused microarray hybridization are performed according to procedures described in Example 2. The QuantArray data results are analyzed according to the procedures described above in Example 2(6).

### 3. Results.

mRNA expression correlates with stratifin protein expression. Increased levels of stratifin mRNA are detected in cell and fluid samples obtained patients suffering from leukemia compared to samples from normal subjects. Cell and fluid samples from patients suffering from leukemia have higher levels of stratifin mRNA expression than do samples from normal subjects.

### Example 12

### Western Blot Analysis of Samples Isolated from Colon Cancer Patients and Normal Subjects

### 1. Patient Samples and Normal Samples

Patient tissues and fluid samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA pool in order to compare them together.

2. Western Blot Analysis of stratifin in Colon and Normal Samples

Samples are prepared as described in Example 1. Western blot analysis is then performed as detailed in Example 1.

### 3. Results.

Stratifin expression is significantly increased in cell and fluid samples obtained from colon cancer patients as compared to cell and fluid samples isolated from normal subjects. All normal subjects show undetectable, or nearly undetectable, levels of stratifin protein expression, while samples obtained from colon cancer patients show detectable levels of stratifin.

### Example 13

### Preparation and Use of Focused Microarray to Detect Stratifin in Samples Obtained From Normal Subjects and Colon Cancer Patients

1. Total RNA Isolation and cDNA Labeling

Patient tissue and fluid samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA pool in order to compare them together.

For colon tissue samples, human tissues are homogenized using the procedure described in Example 2. RNA is isolated and prepared according to procedures described in Example 2.

First strand cDNA labeling, cDNA digestion, capture probe preparation and focused microarray preparation are accomplished using procedures described in Example 2. In addition, quality control and focused microarray hybridization are performed according to procedures described in Example 2. The QuantArray data results are analyzed according to the procedures described above in Example 2(6).

### 2. Results.

mRNA expression correlates with stratifin protein expression. Increased levels of stratifin mRNA are detected in cell and fluid samples obtained patients suffering from colon cancer as compared to normal subjects. Cell and fluid samples from patients suffering from colon cancer have higher levels of stratifin mRNA expression than do samples from normal subjects.

### Example 14

### Western Blot Analysis of Samples Isolated from Prostate Patients and Normal Subjects

### 1. Patient Samples and Normal Samples

Patient tissues and fluid samples are obtained from Asterand, Inc. (Detroit, Mich.), Clinomics Biosciences, Inc (Watervliet, N.Y.) and Biochain Institute, Inc. (Hayward, Calif.). stratifin Each patient included in the study is screened against the same normal total RNA pool in order to compare them together.

### 2. Western Blot Analysis of Stratifin in Prostate and Normal Samples

Samples are prepared as described in Example 1. Western blot analysis is then performed as detailed in Example 1.

Stratifin expression is significantly increased in cell and fluid samples obtained from tumor patients as compared to cell and fluid samples isolated from normal subjects. All normal subjects show undetectable, or nearly undetectable, levels of stratifin protein expression, while several samples obtained from prostate cancer patients show detectable levels of stratifin.

### Example 15

### Preparation and Use of Focused Microarray to Detect Stratifin in Samples Obtained From Normal Subjects and Prostate Cancer Patients

1. Total RNA Isolation and cDNA Labeling

Patient tissue and fluid samples are obtained from Asterand, Inc. (Detroit, Mich.), Clinomics Biosciences, Inc (Watervliet, N.Y.) and Biochain Institute, Inc. (Hayward, Calif.). stratifin Each patient included in the study is screened against the same normal total RNA pool in order to compare them together.

For prostate tissue samples, human tissues are homogenized using the procedure described in Example 2. RNA is isolated and prepared according to procedures described in Example 2.

First strand cDNA labeling, cDNA digestion, capture probe preparation and focused microarray preparation are accomplished using procedures described in Example 2. In addition, quality control and focused microarray hybridization are performed according to procedures described in Example 2. The QuantArray® data results are analyzed according to the procedures described above in Example 2(6).

### 2. Results.

Increased mRNA expression correlates with stratifin protein expression. Increased levels of stratifin mRNA are detected in cell and fluid samples obtained patients suffering from prostate cancer as compared to normal subjects. Cell and fluid samples from patients suffering from prostate cancer have higher levels of stratifin mRNA expression than do samples from normal subjects.

### Example 16

### Classification of Cell Samples Isolated from Breast Normal Subjects and Breast Carcinoma Patients Using Quantitative RT-PCR

### 1. Patient Samples and Normal Samples

Patient material was purchased from Asterand, Inc. (Detroit, Mich.), Clinomics Biosciences, Inc (Watervliet, N.Y.) and Biochain Institute, Inc. (Hayward, Calif.). Seventy-nine patients and sixty-one were tested in Q-PCR.

The level of stratifin expression was identified in breast carcinoma patients and normal subjects utilizing the same techniques as in Example 5. The ratios were generated using the calculations disclosed in Example 5.

### 2. Results

As shown in FIG. 6, increased stratifin expression is identified in certain breast carcinoma patients as compared to normal subjects.

### EQUIVALENTS

Those skilled in the art will recognize, or be able to ascertain, using no more than routine experimentation, numerous equivalents to the specific compositions and procedures described herein. Such equivalents are considered to be within the scope of this invention, and are covered by the following claims.

I claim:

1. A method for detecting a neoplasm comprising
a) obtaining a potentially neoplastic test cell sample and a non-neoplastic control cell sample;
b) detecting a level of stratifin expression in the test cell sample;
c) detecting a level of stratifin expression in the control cell sample;
d) comparing the level of stratifin expression in the test cell sample to the level of stratifin expression in the control cell sample,
the test cell sample being neoplastic if the level of stratifin expression in the test cell sample is detectably greater than the level of stratifin expression in the control cell sample.

2. The method of claim 1, wherein detecting the level of expression of stratifin comprises isolating a cytoplasmic fraction from the test cell sample and from the control cell sample, and then separately detecting the level of expression of stratifin in these cytoplasmic fractions.

3. The method of claim 1, wherein the level of expression of stratifin protein is detected by contacting the test cell sample and the control cell sample with a stratifin-specific protein binding agent of an antibody or stratifin-binding portions thereof.

4. The method of claim 3, wherein the stratifin-specific protein binding agent is an antibody or stratifin-binding portions thereof.

5. The method of claim 3, wherein the protein binding agent is immobilized on a solid support.

6. The method of claim 1, wherein the level of expression of stratifin RNA is detected by contacting the test cell sample and the control cell sample with a stratifin RNA-specific nucleic acid binding agent.

7. The method of claim 6, wherein the nucleic acid binding agent is immobilized on a solid support.

8. The method of claim 1, wherein the level of stratifin expression in the test cell sample is at least 1.5 times greater than the level of stratifin expression in the control cell sample.

9. The method of claim 1, wherein the level of stratifin expression in the test cell sample is at least 2 times greater than the level of stratifin expression in the control cell sample.

10. The method of claim 1, wherein the level of stratifin expression in the test cell sample is at least 4 times greater than the level of stratifin expression in the control cell sample.

11. The method of claim 1, wherein the level of stratifin expression in the test cell sample is at least 6 times greater than the level of stratifin expression in the control cell sample.

12. The method of claim 1, wherein the level of stratifin expression in the test cell sample is at least 8 times greater than the level of stratifin expression in the control cell sample.

13. The method of claim 1, wherein the level of stratifin expression in the test cell sample is at least 10 times greater than the level of stratifin expression in the control cell sample.

14. The method of claim 1, wherein the level of stratifin expression in the test cell sample is at least 20 times greater than the level of stratifin expression in the control cell sample.

15. The method of claim 1, wherein the test cell sample is isolated from a tissue of a patient suffering from a metastasized lung neoplastic disease.

16. The method of claim 1, wherein the test cell sample is isolated from a patient suffering from non-small cell lung carcinoma.

17. A method for diagnosing cancer in a subject comprising:
a) obtaining a potentially neoplastic test fluid sample from the subject, and obtaining a non-neoplastic control fluid sample;
b) detecting a level of stratifin expression in the test fluid sample;
b) detecting a level of stratifin expression in the control fluid sample; and
c) comparing the level of stratifin expression in the test fluid sample to the level of stratifin expression in the control fluid sample, the level of stratifin expression in the test fluid sample is greater than the level of stratifin expression in the control fluid sample is indicative of the presence of cancer in the subject.

18. The method of claim 17, wherein detecting the level of stratifin expression comprises isolating cytoplasmic fractions from the test fluid sample and from the control fluid sample, and separately detecting the level of stratifin expression in the cytoplasmic fractions.

19. The method of claim 17, wherein the detecting steps comprise detecting levels of stratifin protein by contacting the test fluid sample and the control fluid sample with a protein binding agent.

20. The method of claim 19, wherein the protein binding agent is an antibody or stratifin binding protein thereof.

21. The method of claim 20, wherein the protein binding agent is immobilized on a solid support.

22. The method of claim 17, wherein the level of stratifin expression is detected by detecting the level of stratifin RNA expression.

23. The method of claim 22, wherein the level of stratifin RNA expression is detected by contacting the test fluid and the non-neoplastic fluid control fluid with a nucleic acid binding agent.

24. The method of claim 22, wherein the nucleic acid binding agent is immobilized on a solid support.

25. The method of claim 17, wherein the level of stratifin expression in the test fluid sample is about 1.5 times greater than the level of stratifin expression in the control fluid sample.

26. The method of claim 17, wherein the level of stratifin expression in the test fluid sample is about 2 times greater than the level of stratifin expression in the control fluid sample.

27. The method of claim 17, wherein the level of stratifin expression in the test fluid sample is about 4 times greater than the level of stratifin expression in the control fluid sample.

28. The method of claim 17, wherein the level of stratifin expression in the test fluid sample is about 6 times greater than the level of stratifin expression in the control fluid sample.

29. The method of claim 17, wherein the level of stratifin expression in the test fluid sample is about 8 times greater than the level of stratifin expression in the control fluid sample.

30. The method of claim 17, wherein the level of stratifin expression in the test fluid sample is about 10 times greater than the level of stratifin expression in the control fluid sample.

31. The method of claim 18, wherein the level of stratifin expression in the test fluid sample is at least 20 times greater than the level of stratifin expression in the control fluid sample.

32. The method of claim 17, wherein the test fluid sample is from a patient suffering from a metastasized lung neoplastic disease.

33. The method of claim 23, wherein the test fluid sample is from a patient suffering from non-small cell lung carcinoma.

34. A kit for diagnosing or detecting neoplasia, comprising a first probe for the detection of stratifin expression and a detectable label.
